(19) United States
(12) Patent Application Publication
(10) Pub. No.: US 2002/0052343 A1
(43) Pub. Date: May 2, 2002

(54) Salicylamides as serine protease inhibitors

(76) Inventors: Darin Arthur Allen, Daly City, CA (US); Danny Peter Claude McGee, San Mateo, CA (US); Jeffrey R. Spencer, South San Francisco, CA (US)

Correspondence Address: Axys Pharmaceuticals, Inc., 180 Kimball Way, South San Francisco, CA, 94080, US

(21) Appl. No.: 09/737,687

(22) Filed: Dec. 14, 2000

Related U.S. Application Data
(60) Provisional application No. 60/170,916, filed on Dec. 15, 1999.

(51) Int. Cl. A61K 31/675; A61K 31/655

(52) U.S. Cl. 514/79; 514/150; 514/231.5; 514/387; 514/461; 514/619; 534/727; 534/751; 534/768; 544/139; 544/165; 548/304.4; 549/491; 564/167

(57) ABSTRACT

The present invention provides novel compounds of Formula I:

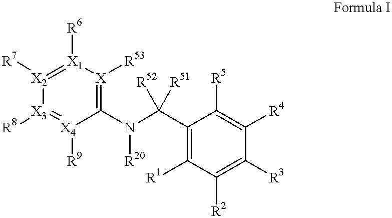

its prodrug forms, or pharmaceutically acceptable salts thereof. The compounds of this invention are inhibitors of serine proteases, Urokinase (uPA), Factor Xa (FXa), and/or Factor VIIa (FVIIa), and have utility as anti cancer agents and/or as anticoagulants for the treatment or prevention of thromboembolic disorders in mammals. The present invention also provides a process for the selective acylation of an amino group.

This application is based on and claims priority from U. S. Provisional Application Ser. No. 60/170,916 filed on Dec. 15, 1999.

### FIELD OF INVENTION

The present invention relates to novel serine protease inhibitors.

### BACKGROUND OF THE INVENTION

One of the most active areas in cancer research is in the field of proteolytic enzymes and their role in the spread of cancer. One class of proteases that plays a significant role in the progression of cancer are the serine proteases, in particular Urokinase-type plasminogen activator (uPA). Inhibitors of uPA have been postulated to be of therapeutic value in treating cancer. Inhibitors of these serine proteases also tend to be inhibitors of the closely related blood-clotting enzymes. One such blood-clotting enzyme is Factor Xa.

Factor Xa (herein after “FXa”), the converting enzyme of pro-thrombin to thrombin, has emerged as an alternative target (to thrombin) for drug discovery for thromboembolic disorders. A variety of compounds have been developed as potential FXa inhibitors.

Kunitada and Nagahara in Current Pharmaceutical Design, 1996, Vol. 2, No.5, report amidinobenzyl compounds as FXa and thrombin inhibitors. Disclosed in U.S. Pat. No. 5,576,343 are aromatic amidine derivatives and salts thereof, as reversible inhibitors of FXa. These compounds comprise amidino substituted indolyl, benzofuranyl, benzothienyl, benzimidazolyl, benzoxazoyl, benzothiazolyl, naphthyl, tetrahydronaphthyl and indanyl groups, attached to a substituted phenyl ring by an alkylene group having from 1 to 4 carbon atoms.

In spite of the above discussed efforts, desirable treatment of cancer and thromboembolic disorders still remains elusive. There is thus a need for new compounds that will be effective in inhibiting serine proteases, such as Urokinase, and blood-clotting enzymes such as FXa. Keeping these needs in mind, the present invention provides novel inhibitors as discussed below.

### SUMMARY OF THE INVENTION

Keeping the above discussed needs in mind, the present invention provides novel salicylamides of Formula I as serine protease inhibitors. Included in the present invention are pharmaceutically acceptable salts of compounds of Formula I, a pharmaceutical composition comprising a pharmaceutically acceptable carrier and a therapeutically effective amount of a compound or a pharmaceutically acceptable salt of a compound of Formula I, a method of treating or preventing a thromboembolic disorder, comprising administering to a patient in need thereof a therapeutically effective amount of a compound of Formula I, and a method for treating cancer in mammals comprising administering a therapeutically effective amount of a compound of Formula I. Also provided by the present invention is a process for selectively acylating an amino group.

### DETAILED DESCRIPTION

Provided by the present invention is a compound of Formula I:

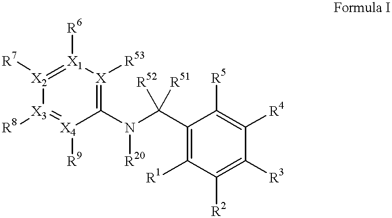

its prodrug form or pharmaceutically acceptable salts thereof, wherein:

R¹ represents OH, COOH, COO—C₁₋₄alkyl, CH₂OR¹⁰, SO₂—OH, O—SO₂—OH, O—SO₂-OC₁₋₄alkyl, OP(O)(OH)₂, or OPO₃C₁₋₄alkyl;

R², R³, R⁴, and R⁵ independently at each occurrence represent H, SH, OR¹⁰, halogen, COOR¹⁰, CONR¹¹R¹², optionally substituted aryl, optionally substituted heterocyclyl, C₄₋₁₄cycloalkyl-C₁₋₄alkyl, C₁₋₄alkyl aryl, optionally substituted C₁₋₁₄ straight chain, branched or cyclo alkyl, NR¹⁰R²⁴, (CH₂)₁₋₄—NR³³R³⁴, (CH₂)₁₋₄—COOR³³, O—(CH₂)₁₋₃—CO—het, O—(CH₂)₁₋₂—NH—CO—aryl, O—(CH₂)₀₋₂—NR¹⁰—CO—NR¹⁰R³³, O—(CH₂)₀₋₂—C(O)— NR³³R³⁴, O—(CH₂)₁₋₄—COOR¹⁰, O—(CH₂)₁₋₃—het—R³², O-optionally substituted cycloalkyl, O—(CH₂)₁₋₄—NR¹⁰—COO—t-butyl, O—(CH₂)₁₋₄—NR¹⁰R³³, O—(CH₂)₁₋₄—NR¹⁰— C(O)—CO₃-alkyl-optionally substituted aryl, O—(CH₂)₀₋₆-optionally substituted aryl, (CH₂)₁₋₄—NH—C(O)O—(CH₂)₁₋₄-PhR¹³R¹⁴, NO₂, O—(CH₂)₀₋₄—C(O)-NH-tetrahydro carboline, SO₃H, CH(OH)COOR¹⁰, NR¹⁰R²⁸, O—(CH₂)₁₋₃-optionally substituted het, CH₂COOCH₃, CH═CH—COOCH₃,

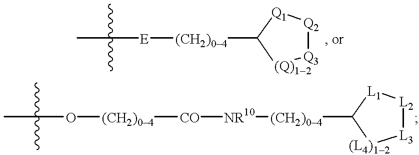

alternatively R² and R³, R³ and R⁴, or R⁴ and R⁵ taken together form

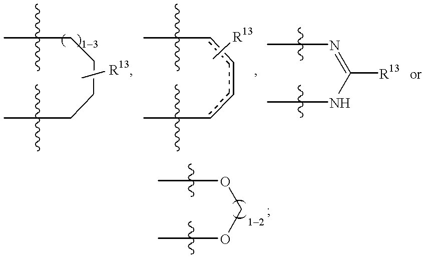

R⁶, R⁹ and R⁵³ independently at each occurrence represents H, halogen, cyano, C₁₋₄alkyl, C₁₋₄halogenated alkyl, NO₂, O—aryl or OR¹¹; alternatively R⁶ and R⁵³ taken together form

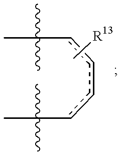

R⁷ and R⁸ independently at each occurrence represent OH, CF₃, H, COOH, NO₂, C₁₋₄alkyl, OC₁₋₄alkyl, or O-aryl, halogen, cyano, or a basic group selected from guanidino, NH(CH═NH)NH₂, C(═NH)N(R¹⁰)₂, C(═NH)—NH—NH₂, C(═O)N(R¹⁰)₂, 2— imidazoline, N-amidinomorpholine, N-amnidino piperidine, 4-hydroxy-N-amidino piperidine, N-amidino pyrrolidine, tetrahydro pyrimidine, C(O)CH₂NH₂, C(O)NHCH₂CN, NHCH₂CN, and thiazolidin-3-yl-methylideneamine; with the proviso that only one of R⁷ and R⁸ represent a basic group;

R¹⁰ independently at each occurrence represents H, (CH₂)₀₋₂-aryl, C₁₋₄halo alkyl, or C₁₋₁₄ straight chain, branched or cyclo alkyl, and alternatively, when one atom is substituted with two R¹⁰ groups, the atom along with the R¹⁰ groups can form a five to 10 membered ring structure;

X₁, X₂, X₃ and X₄ independently at each occurrence represent a carbon or a nitrogen atom;

R¹¹ and R¹² independently at each occurrence represent H or C₁₋₄alkyl;

R¹³ represents H, OH, OC₁₋₄alkyl, OAr, OC₅₋₁₀cycloalkyl, OCH₂CN, O(CH₂)₁₋₂NH₂, OCH₂COOH, OCH₂COO—C₁₋₄alkyl or

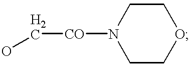

R²⁰ represents H or OH;

R²⁴ represents R¹⁰, (CH₂)₁₋₄-optionally substituted aryl, (CH₂)₀₋₄OR¹⁰, CO—(CH₂)₁₋₂— N(R¹⁰)₂, CO(CH₂)₁₋₄—OR¹⁰, (CH₂)₁₋₄—COOR¹⁰, (CH₂)₀₋₄—N(R¹⁰)₂, SO₂R¹⁰, COR¹⁰, CON(R¹⁰)₂, (CH₂)₀₋₄-aryl—COOR¹⁰, (CH₂)₀₋₄—aryl—N(R¹⁰)₂, or (CH₂)₁₋₄—het—aryl;

R²⁸ represents (CH₂)₁₋₂—Ph—O—(CH₂)₀₋₂—het—R³⁰, C(O)—het, CH₂—Ph—CH₂—het—(R³⁰)₁₋₃;

(CH₂)₁₋₄-cyclohexyl—R³¹, CH₂—Ph—O—Ph—(R³⁰)₁₋₂, CH₂—(CH₂OH)—het—R³⁰, CH₂—Ph—O— cycloalkyl-R³¹, CH₂—het—C(O)—CH₂—het—R³⁰, or CH₂—Ph—O—(CH₂)—O—het—R³⁰;

R³⁰ represents SO₂N(R¹⁰)₂, H, NHOH, amidino, or C(═NH)CH₃;

R³¹ represents R³⁰, amino-amidino, NH—C(═NH)CH₃ or R¹⁰;

R³² represents H, C(O)—CH₂—NH₂, or C(O)—CH(CH(CH₃)₂)—NH₂;

R³³ and R³⁴ independently at each occurrence represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄—N(R¹⁰)₂, (CH₂)₁₋₄—OH, (CH₂)₁₋₄—SO₂—N(R¹⁰)₂;

alternatively, R³³ and R³⁴ along with the nitrogen atom that they are attached to forms a 4 to 14 atom ring structure selected from tetrahydro-1H-carboline; 6,7-Dialkoxyoxy-2-substituted 1,2,3,4-tetrahydro-isoquinoline,

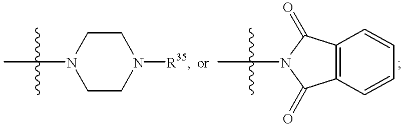

R³⁵ represents R¹⁰, SO₂—R¹⁰, COR¹⁰, or CONHR¹⁰;

E represents a bond, S(O)₀₋₂, O or NR¹⁰;

Q, Q¹, Q², Q³, L¹, L², L³ and L⁴ independently at each occurrence represent N-natural or unnatural amino acid side chain, CHR¹⁰, O, NH, S(O)₀₋₂, N—C(O)—NHR¹⁰, SO₂— N(R¹⁰)₂, N—C(O)—NH—(CH₂)₁₋₄—R²⁶, NR¹⁰, N—heteroaryl, N—C(═NH)—NHR¹⁰, or N—C(═NH)C₁₋₄alkyl;

R²⁶ represents OH, NH₂, or SH;

R⁵¹ and R⁵² independently represent COOH, CH₂OH, CH₂COOH, COOR, CH₂COOR, alkyl or CO—NH₂; alternatively

R⁵¹ and R⁵² taken together represent ═O, ═S, ═CH₂ or ═NR¹⁰;

R⁵³ represents H, halogen, cyano, C₁₋₄alkyl, C₁₋₄halogenated alkyl, NO₂, O-aryl or OR¹¹;

with the proviso that at least two of X₁, X₂, X₃ and X₄ represent a carbon atom, and when any of X₁, X₂, X₃ and X₄ represent a nitrogen atom the corresponding substituent does not exist.

In a preferred embodiment of the present invention is provided a compound of Formula I wherein, R¹ represents OH or COOH; R²⁰ represents H; R⁵¹ and R⁵² taken together form ═O; and X₁, X₂, X₃, and X₄ represent C. Another preferred embodiment provides a compound wherein, R² represents halo, H, NH—CO—Ph, i-propyl, OH, OCH₃, OC₂H₅, CH(OH)COOH, O—I-propyl, SO₃H, NH₂, CH(OH)COOC₁₋₂alkyl, CH₃, NO₂ or Ph;

R³ represents H, OH, NH₂ OC₁₋₄alkyl, C₁₋₄alkyl, NHCH₃, O—(CH₂)₁₋₃—OCO—C₁₋₂alkyl, NH—C(O)C₁₋₂alkyl, O—(CH₂)₁₋₂—CO—NH₂, Ph, NHCOCF₃, N═CH—N(CH₃)₂, O—CH₂—CO—NH—(CH₂)₁₋₃—Ph,

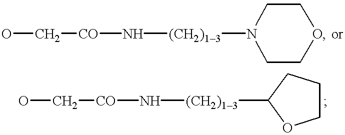

R⁴ represents H, C₁₋₄alkyl, halogen, i-propyl, OH, NH₂ 3-nitro-phen-1-yl, NH—CO— CH₃, CH₂—NH—(CH₂)₃—Ph, 2,4-difluoro-phen-1-yl, NHCOCF₃, benzo[1,3]dioxol-5-yl,

4-Carbamimidoyl-phenylazo, 3-Hydroxy-4-carboxyl-phenylsulfanyl; 1,3-Dioxo-indan-2-yl, or toluene-4-sulfonylamino;

R⁵ represents H or OH;

alternatively, R² and R³, R³ and R⁴, or R⁴ and R⁵ can be taken together to form

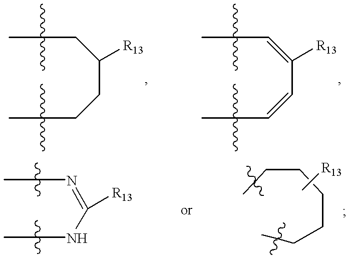

R⁶ represents H;

R⁷ represents C(═NH)—NH₂ or NH—C(═NH)—NH₂;

R⁸ represents H or halogen; and

R⁹ represents H.

A further preferred embodiment provides a compound wherein, R² represents halo, H, NH—CO—Ph, i-propyl, OH, CH₃, or NO₂;

R³ represents H, OH, NH₂ OC₁₋₂alkyl, C₁₋₄alkyl, O—(CH₂)₁₋₃—OCO—C₁₋₂alkyl, NH— C(O)CH₃, O—CH₂—CO—NH₂, Ph, NHCOCF₃, N═CH—N(CH₃)₂, O—CH₂—CO—NH—(CH₂)₂—Ph;

R⁴ represents H, CH₃, methoxy, halogen, i-propyl, 3-nitro-phen-1-yl, NHCOCF₃, benzo[1,3]dioxol-5-yl, NHCOCH₃, 4-Carbamimidoyl-phenylazo, 3-Hydroxy-4-carboxyl-phenylsulfanyl or 1,3-Dioxo-indan-2-yl;

alternatively, R² and R³, R³ and R⁴, or R⁴ and R⁵ can be taken together to form

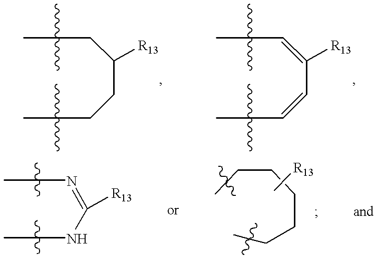

R¹³ represents C₁₋₂alkyl, OH, O(CH₂)₁₋₂—NH₂, H, or

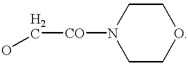

Particularly preferred compounds of the present invention are:

N-(4—Carbamimidoyl-phenyl)-2-hydroxy-3-iodo-5-methyl-benzamide;

3,5-Dibromo-N-(4-carbamimidoyl-phenyl)-2,4-dihydroxy-benzamide;

5-Bromo-N-(4-carbamimidoyl-phenyl)-2,4-dihydroxy-3-iodo-benzamide;

3-Hydroxy-naphthalene-2-carboxylic acid (6-guanidino-pyridin-3-yl)-amide; and

3-Hydroxy-7-methoxy-naphthalene-2-carboxylic acid (4-guanidino-phenyl)-amide.

Another aspect of the present invention provides a pharmaceutical composition comprising a pharmaceutically acceptable carrier and a therapeutically effective amount of (i) a compound; or (ii) a pharmaceutically acceptable salt of a compound of Formula I. Also provided by the present invention is a method of treating or preventing a thromboembolic disorder, comprising administering to a patient in need thereof a therapeutically effective amount of a compound of Formula I, or a pharmaceutically acceptable salt thereof.

In yet another aspect of the present invention is provided a process for selectively acylating an amino group, said process comprising treating a molecule comprising an amino group with an acylating agent in the presence of an acetamide to yield a compound with an acylated amino group. A preferred embodiment provides a process wherein the amino group is selectively acylated in the presence of another acylatable group. Yet another preferred embodiment provides a process wherein the acylatable group is selected from an optionally substituted amino ketone, alkyl amidino, alkyl guanidino, C(═NH)NH—NH₂, aryl—(CH₂)₀₋₄—NHR¹⁰, amidino and guanidino; the acylating agent comprises an acid halide group; and wherein the acetamide is an alkyl or dialkyl acetamide.

A further preferred embodiment provides a process wherein the acetamide is selected from a group consisting of DMA, diethyl acetamide, dimethyl propionamide, diethyl propionamide and N-methylpyrrolidinone; the process is carried out at a temperature ranging from about 25° C. to about 50° C.; and wherein the acylating agent is a protected salicylic acid chloride selected from acetic acid 2-chlorocarbonyl-phenyl ester and 2-benzyloxy-benzoyl chloride.

Experimental

Novel compounds of the present invention can be prepared by the synthetic schemes outlined below:

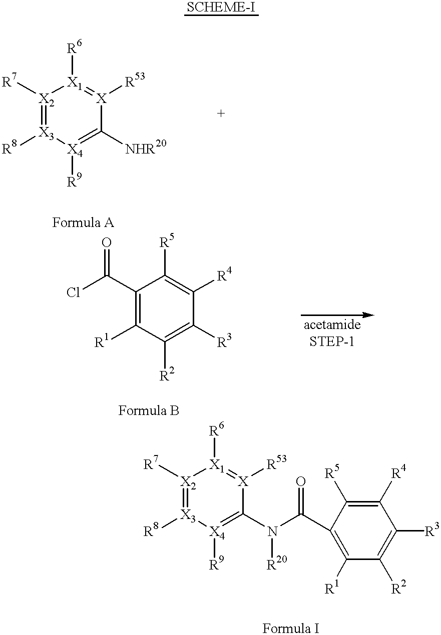

Step-1

A mixture of a compound of Formula A (1 eq.), a compound of Formula B (1.2 eq.) and dimethyl acetamide (DMA) is stirred at ambient temperature from about 30 minutes to about 2 hours, or until a TLC analysis indicates absence of the compound of Formula A. The reaction mixture then is diluted with ether or water leading to the formation of a precipitate of a compound of Formula I. This precipitate is isolated and dried. Structural confirmation and compound identification is accomplished by techniques such as proton NMR (¹H NNM), mass spectral analysis (MS) and elemental analysis.

Formula I (R¹═OH)

Conversion of Formula I compounds, where R¹ is O-acetyl, to Formula I compounds, where R¹ is OH, is accomplished by treating a compound of Formula I with a base, preferably aqueous ammonium hydroxide. The reaction mixture is initially clear but formation of a yellowish precipitate indicates the conversion of an O-acetyl group to a hydroxy group. This conversion is generally quantitative. The precipitate is isolated and dried to yield the corresponding compound of Formula I, where R¹ is OH.

Acid Salts

Acid salts of compound of Formula I can be formed by stirring a compound of Formula I, having at least one amino center, with an acid, preferably a mineral acid such as HCl. This affords the corresponding acid salt of a compound of Formula I as a solid. The solid is isolated and dried. Structural identification is accomplished using techniques such as (¹H NMR), MS and elemental analysis.

Synthesis of Starting Materials

Some of the compounds of Formula A and Formula B can be purchased from commercial sources such as Aldrich Chemicals and Lancaster. Compounds of Formula A and Formula B can also be prepared by synthetic methods known to one skilled in the art. Thus compounds of Formula B can be synthesized as described below.

Synthesis of Compounds of Formula B:

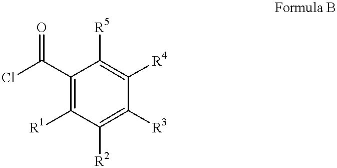

Compounds of Formula B are acid chlorides which can be synthesized by dissolving an appropriate carboxylic acid in an appropriate solvent, for example ethyl acetate (EtOAc) with a catalytic amount of DMF, and treating this mixture with about 1.5 equivalents of oxalyl chloride. The resulting reaction mixture is stirred at ambient temperature for about 30 minutes. The solvent is evaporated to obtain a compound of Formula B. These compounds of Formula B can be used without further purification.

The acetylated carboxylic acid used above can, in turn, be prepared by acetylating the corresponding hydroxy carboxylic acid, e.g., salicylic acid. The procedure comprises combining a suspension of the hydroxy carboxylic acid in acetic anhydride with catalytic amount of acid, e.g., sulfuric acid and agitating this mixture from about 1 to about 3 hours at ambient temperature. The acetylated carboxylic acid falls out of the solution as a solid. This acetylated carboxylic acid then is used as described above.

Scheme II

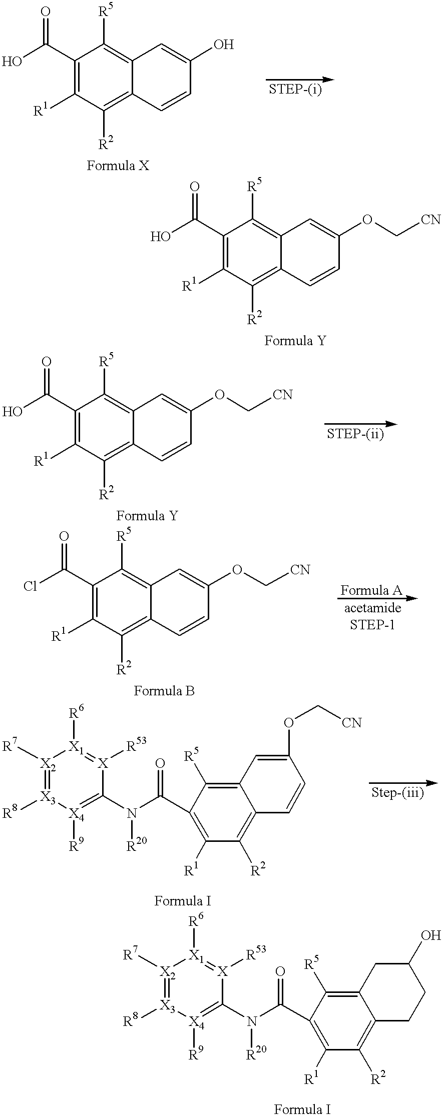

Step-(i)

A compound of Formula X (500 mg, 2.5 mmol) was mixed with DMF (5 ml) and 60% sodiumhydroxide (0.32 g) to form a mixture. The mixture then was stirred for about 30 minutes. The stirred mixture was combined with chloroacetonitrile (0.17 ml, 1.1 eq.) and the new reaction mixture was stirred for about 1 hour followed by dilution with 1 N HCl to form a precipitate. The precipitate was isolated and dried to yield a compound of Formula Y.

Step-(ii)

Compounds of Formula B are acid chlorides which can be synthesized by dissolving an appropriate corresponding carboxylic acid in an appropriate solvent, for example ethyl acetate (EtOAc) with a catalytic amount of DMF, and treating this mixture with about 1.5 equivalents of oxalyl chloride. The resulting reaction mixture is stirred at ambient temperature for about 30 minutes. The solvent is evaporated to obtain a compound of Formula B. These compounds of Formula B can be used without further purification.

Step-1

A mixture of a compound of Formula A (1 eq.), a compound of Formula B (1.2 eq.) and dimethyl acetamide (DMA) was stirred at ambient temperature from about 30 minutes to about 2 hours, or until a TLC analysis indicates absence of the compound of Formula A. The reaction mixture then was diluted with ether or water leading to the formation of a precipitate of a compound of Formula I. This precipitate was isolated and dried. Structural confirmation and compound identification was accomplished by techniques such as proton NMR (¹H NMR), mass spectral analysis (MS) and elemental analysis.

Step-(iii)

A compound of Formula I (Ex. 168 ) was combined with a mixture of methanol and 1 N HCl followed. The resulting mixture was further combined with Platinum oxide and this mixture was agitated under hydrogen at 35 PSI for about 1 hour. The agitated mixture was filtered and concentrated to yield an oily substance. The oily substance was purified by preparative HPLC eluting with a gradient of 10-90% solvent A in solvent B (The solvent A was 20 mm HCl, solvent B was acetonitrile) to yield a compound of Formula I (Ex. 175).

Compounds of Formula I wherein R²═SO₃H

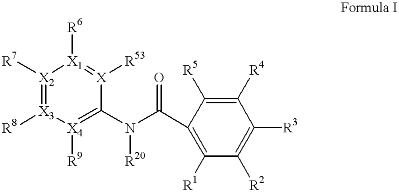

A compound of Formula I (R²═H) (100 mg, 0.31 mmol) was dissolved in concentrated sulfuric acid (2 ml) and then mixed with a sulfur trioxide-N,N-dimethylformamide complex (120 mg, 0.78 mmol). The resulting solution was heated at about 50° C. for about 10 minutes, and then diluted with water to yield a precipitate. The precipitate was isolated and dried to yield a compound of Formula I wherein R²═SO₃H (Ex. 173).

Synthesis of Compounds wherein R²═OH or NH₂. A compound of Formula I (R²═H) (120 mg, 0.37 mmol) was suspended in water (6 ml) and the suspension was treated with fuming nitric acid (0.5 mL). The resulting mixture was stirred from about 8 to about 16 hours and the solids were isolated by filtration. The solids then were dissolved in a mixture of methanol (10 mL) and 1 N HCl (1 mL), the solution was combined with Palladium(II)hydroxide catalyst (20%) and the resulting reaction mixture was agitated in an atmosphere of hydrogen for about 12 hours. The agitated reaction mixture was filtered through celite and the filtrate was concentrated under reduced pressure to yield a residue. The residue was purified and the two components of the residue were separated using reverse phase HPLC to yield two compounds of Formula I wherein R²═OH and NH₂ respectively.

### EXAMPLES

Listed in TABLES-I, II and III are compounds which were synthesized using the procedures discussed above.

TABLE I

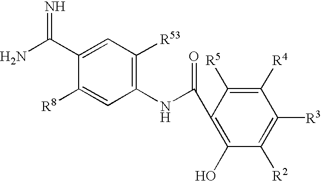

R⁸ and R⁵³ represent H, unless noted otherwise.

| Ex. | R² | R³ | R⁴ | R⁵ | |
|---|---|---|---|---|---|
| 1 | I | H | CH₃ | H | |
| 2 | Br | OH | Br | H | |
| 3 | I | OH | Br | H | |
| 4 | I | NH₂ | I | H | |
| 5 | Br | H | CH₃ | H | |
| 6 | Br | NH₂ | Br | H | |
| 7 | I | H | CH₃ | H | R⁸ = F |
| 8 | I | H | F | H | |
| 9 | Br | OH | H | H | |
| 10 | H | 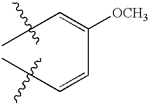 | | H | |
| 11 | H | 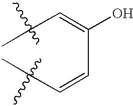 | | H | |
| 12 | I | H | Cl | H | |
| 13 | H | 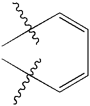 | | H | |
| 14 | Br | H | F | H | |
| 15 | Cl | H | H | H | |
| 16 | H | OC₂H₅ | H | H | |
| 17 | Br | OCH₃ | Br | H | |
| 18 | H | NH₂ | H | H | |
| 19 | H | CH₃ | H | H | |
| 20 | 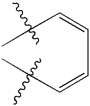 | | H | H | |
| 21 | Br | H | Br | H | |
| 22 | H | OCH₂CH₂O | H | H | |
| | | C(O)CH₃ | | | |
| 23 | Br | CH₃ | Br | H | |
| 24 | CH(CH₃)₂ | H | CH(CH₃₎₂ | H | |
| 25 | OH | H | H | H | |
| 26 | H | H | H | OH | |
| 27 | CH₃ | H | H | H | |
| 28 | Cl | H | Cl | H | |
| 29 | Br | H | benzo[1,3]di | H | |
| | | | oxol-5-yl | | |
| 30 | NO₂ | H | NHC(O)CF₃ | H | |
| 31 | CH(CH₃)₂ | H | H | H | |
| 32 | H | 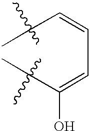 | | H | |
| 33 | H | 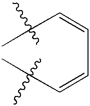 | | H | R⁸ = F |
| 34 | H | OCH₃ | H | H | |
| 35 | H | NHC(O)CH₃ | H | H | |
| 36 | H | H | NH₂ | H | |
| 37 | H | H | CH₃ | H | |
| 38 | H | H | H | H | |
| 39 | H | OCH₂C(O)NH₂ | H | H | |
| 40 | H | H | OCH₃ | H | |
| 41 | H | OH | H | H | |
| 42 | H | NHC(O)CF₃ | H | H | |
| 43 | H | OH | H | OH | |
| 44 | H | N═CH— | H | H | |
| | | N(CH₃)₂ | | | |
| 45 | H | H | I | H | |
| 46 | H | (3-phenyl- | H | H | |
| | | propylcarbamo | | | |
| | | yl)-methoxy | | | |
| 47 | Br | H | 3-nitro-phenyl | H | |
| 48 | H | H | 4- | H | |
| | | | carbamimido | | |
| | | | yl-phenylazo | | |
| 49 | H | OH | Br | H | |
| 50 | H | phenethylcarb | H | H | |
| | | amoyl- | | | |
| | | methoxy | | | |
| 51 | H | H | NHC(O)CH₃ | H | |
| 52 | H | benzylcarbam | H | H | |
| | | oyl-methoxy | | | |
| 53 | H | Cl | H | H | |
| 54 | H | H | (3-phenyl- | H | |
| | | | propylamino)- | | |
| | | | methyl | | |
| 55 | H | H | F | H | |
| 56 | H | H | 2,4- | H | |
| | | | difluorophenyl- | | |
| | | | 1-yl | | |
| 57 | H | H | 3-(4- | H | |
| | | | carbamimidoyl- | | |
| | | | phenylcarbamoyl)- | | |
| | | | 4-hydroxy- | | |
| | | | phenylsulfanyl | | |
| 58 | H | (2-morpholin- | H | H | |
| | | 4-yl-ethyl- | | | |
| | | carbamoyl)- | | | |
| | | methoxy | | | |
| 59 | H | H | Cl | H | |
| 60 | H | H | Br | H | |
| 61 | H | H | benzo[1,3]di | H | |
| | | | oxol-5-yl | | |
| 62 | H | [(tetrahydro- | H | H | |
| | | furan-2- | | | |
| | | ylmethyl)- | | | |
| | | carbamoyl]- | | | |
| | | methoxy | | | |
| 63 | 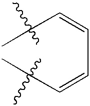 | | OH | H | |
| 64 | H | H | 2-carboxy-4- | H | |
| | | | mercaptoyl- | | |
| | | | phenol | | |
| 65 | Ph | H | H | H | |
| 66 | H | H | H | H | R⁵³ = CH₃ |
| 67 | H | H | 1,3-dioxo- | H | |
| | | | 1,3-dihydro- | | |
| | | | isoindol-2-yl | | |
| 68 | H | H | NHC(O)CF₃ | H | |
| 69 | H | H | toluene-4- | H | |
| | | | sulfonylamino | | |
| 70 | H | H | 3-nitrophen-1- | H | |
| | | | yl | | |
| 71 | I | H | CH₃ | H | R⁸ = F |
| 72 | H | O(CH₂)₅COOC₂H₅ | H | H | |
| 73 | H | O(CH₂)₅COOH | H | H | |
| 74 | NH₂ | H | H | H | |
| 75 | H | 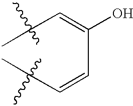 | | H | |
| 76 | 4-cyano- | H | H | H | |
| | benzoylamino | | | | |
| 77 | NHC(O)-Ph | H | H | H | |
| 78 | H | OCH₂Ph | H | H | |
| 79 | H | 4-ethoxy- | H | H | |
| | | carbonyl- | | | |
| | | cyclohexyloxy | | | |
| 80 | I | H | CH₃ | H | R⁵³ = Cl |
| 81 | H | 4-Carbamimi- | OH | H | |
| | | doyl-phenyl | | | |
| | | carbamoyl | | | |

Listed below is the proton NMR (¹H NMR) and Mass spectral data for compounds listed in TABLE-I.

### Ex. 1.

¹H NMR (DMSO- d₆) δ12.42 (s, 1H), 10.91 (s, 1H), 9.33 (s, 2H), 9.02 (s, 2H), 7.98-7.85 (m, 6H), 2.30 (s, 3H)

Mass Spec (M+1)=396

### Ex. 2

¹H NMR (DMSO-d₆) δ10.8(br, 2H), 9.3(br s, 2H), 8.9(br s, 2H), 8.4(s, 1H), 7.85(m, 4H)

Mass Spec (M+1)=429.6

### Ex. 4

¹H NMR (DMSO-d₆) δ9.28 (s, 2H), 8.94 (s, 2H), 8.50 (s, 1H), 7.92 (d, 2H, J=8.91), 7.85 (d, 2H. J =8.91), 5.90 (s, 2H).

### Ex. 5

¹H NMR (DMSO-d₆) δ12.11 (s, 1H), 10.92 (s, 1H), 9.31 (s, 2H), 9.03 (s, 2H), 7.97-7.85 (m, 5H), 7.66 (s, 1H), 2.30 (s, 3H).

Mass Spec (M+1)=347.7

### Ex. 6

¹H NMR (DMSO-d₆) δ10.60 (s, 1H), 9.28 (s, 2H), 8.97 (s, 2H), 8.38 (s, 1H), 7.93 (d, 2H, J=8.91), 7.85 (d, 2H. J=8.66).

### Ex. 8

¹H NMR (DMSO-d₆) δ9.31 (s, 2H), 8.98 (s, 2H), 8.07 (d, 1H, J=9.65), 7.99-7.91 (m, 3H), 7.85 (d, 2H, J=8.66).

Mass Spec (M+1)=399.7

### Ex. 10

¹H NMR (DMSO-d₆) δ9.2(br), 8.25(s, 1H), 7.85(br s, 4H), 7.31(d, 1H, J=9 Hz), 7.06(d, 1H, J=2.2 Hz), 6.87(dd, 1H, J=2.5, 8.8 Hz), 6.74(s, 1H), 3.78(s, 3H)

Mass Spec (M+1)=336.6

### Ex. 11

¹H NMR (DMSO-d₆) δ8.28(m, 1H), 8.16(m, 1H), 7.90(m, 2H), 7.83(m, 2H), 7.5(m, 2H), 6.96(m, 2H), 6.74(s, 1H)

Mass Spec (M+1)=321.9

### Ex. 12

¹H-NMR (DMSO-d₆) δ11.87 (s, 1H), 10.42 (s, 1H), 9.81 (s, 1H), 7.89 (d, 1H, J=7.97 Hz), 7.78 (d, 2H, J=8.09 Hz), 7.43 (s, 3 H), 7.22 (d, 2H, J=8.56 Hz), 6.80-6.70 (m, 1S 2H), 2.28 (s, 3H).

Mass Spec (M+1)=284.9

### Ex. 13

¹H NMR (DMSO-d₆) δ11.2(br s, 1H), 10.95(br s, 1H), 9.3(br s, 2H), 9.0(br s, 2H), 8.45(s, 1H), 8.05-7.9(m, 5H), 7.8(d, 1H), 7.55(t, 1H), 7.35(m, 2H)

Mass Spec (M+1)=306.3

### Ex. 14

¹H NMR (DMSO-d₆) δ10.96 S, 1H), 9.31 (s, 2H), 8.98 (s. 2H), 8.03 (d, 1H, J=8.66), 7.95 (d, 2H, J=8.42),7.88-7.85 (m, 3H).

Mass Spec (M+1)=353.6

### Ex. 15

¹H NMR (DMSO-d₆) δ7.89 (d, 2H, J=8.91 Hz), 7.79 (d, 2H, J=8.91 Hz), 7.65 (dd, 1H, J=1.98, 7.92 Hz), 7.19 (dd, 1H, J=1.98, 7.43 Hz), 6.11 (t, 1H, J=7.67 Hz).

Mass Spec (M+1)=289.7

### Ex. 16

¹H NMR (DMSO-d₆) δ12.08 (s, 1H), 10.56 (s, 1H), 9.27 (s, 2H), 8.95 (s, 2H), 7.99 (d, 2H, J=8.97 Hz), 7.94 (s, 1H), 7.85 (d, 2H, J=8.97 Hz), 6.59-6.53 (m, 2H), 4.07 (q, 2H, J=6.86 Hz), 1.43 (t, 3H, J=6.86 Hz).

Mass Spec (M+1) =299.9

### Ex. 17

¹H NMR (DMSO-d₆) δ10.30(s, 1H), 9.23 (s, 2H), 8.91 (s, 2H), 7.92 (d, 2H, J=8.42 Hz), 7.83-7.74 (m, 3H), 6.19 (d, 1H, J=8.91 Hz), 6.10 (s, 1H).

Mass Spec (M+1)=270.7

### Ex. 18

¹H NMR (DMSO-d₆) δ9.31(s, 2H), 8.98 (s, 2H), 8.47 (s, 1H), 7.95-7.86 (m, 4H), 3.86 (s, 3H).

Mass Spec (M+1)=443.8

### Ex. 19

¹H NMR (DMSO—d₆) δ11.7(br s, 1H), 10.65(br s, 1H), 9.4(br s, 2H), 9.05(br s, 2H), 7.9(m, 5H), 6.8(m, 2H), 2.3(s, 3H).

### Ex. 23

¹H NMR (DMSO-d₆) δ12.8(br s, 1H), 11.05(s, 1H), 9.3(br s, 2H), 9.08(br s, 2H), 8.4(d, 1H, J=2.2 Hz), 7.89(m, 4H), 3.55( s, 3H)

Mass Spec (M+1)=427.6

### Ex. 30

¹H NMR (DMSO-d₆) δ11.6(br s, 1H), 11.5(br s, 1H), 9.3(br s, 2H), 8.9(br s, 2H), 8.5(s, 1H), 8.3(s, 1H), 7.9(m, 4H)

Mass Spec (M+1)=411.8

### Ex. 32

¹H NMR (DMSO-d₆) δ9.2(br, 4H), 8.26(d, 1H, J=3.3 Hz), 7.87(br s, 4H), 7.11(d, 1H, J=3.2 Hz), 7.02(d, 1H, J=3 Hz), 6.73(m, 1H), 6.60(m, 1H)

Mass Spec (M+1)=321.9

### Ex. 36

¹H NMR (DMSO-d₆) δ10.75(s, 1H), 10.25(br, 3H), 9.35(br s, 2H), 9.05(br s, 2H), 7.95(m, 4H), 7.85(d, 1H), 7.45(d, 1H), 7.2(d, 1H)

Mass Spec (M+1)=270.8

### Ex. 38

### N-(4-carbamimidoyl-phenyl)-2-hydroxy-benzamide

A solution of 4-aminobenzonitrile (1 g; 7.57 mmol) in THF (25 mL) was combined with acetylsalicyloyl chloride (11.5 g; 1 eq.) and Et₃N (2 mL). This mixture was agitated for 8-12 hours and then diluted with ethyl acetate (50 mL). The diluted mixture was washed in succession with 1 M HCl solution (15 mL), brine (50 mL), dried (MgSO₄) and concentrated under reduced pressure to yield a yellow colored oily residue

Purification of the oily residue by flash chromatography yielded 4-(2-acetoxybenzamido)-benzonitrile (0.9g).

The above 4-(2-acetoxybenzamido)-benzonitrile (0.9 g) was dissolved in a 1:3 mixture of dioxane:ethyl acetate (15 mL) and the resulting mixture was cooled to a temperature of from about 0° C. to about 15° C. The cold reaction mixture was saturated with gaseous HCl, the reaction vessel was sealed and the reaction mixture was agitated from about 8 to about 12 hours. The reaction mixture was concentrated under reduced pressure to yield a solid. This solid was dissolved in a 2 M ammonia solution in ethanol and the resulting mixture was agitated in a sealed reaction vessel from about 8 to about 16 hours. The reaction mixture was concentrated under reduced pressure to yield an oily residue. The oily residue was purified using purification techniques known to one skilled in the art, for example BPLC, to yield N-(4-Carbamimidoyl-phenyl)-2-hydroxy-benzamide (27 mg).

¹H NMR (DMSO-d₆) δ11.58(br. S, 1H), 10.75(br S, 1H), 9.26(br S, 2H), 8.94(br S, 2H), 7.93(dd, 2H, J=8.8, 1.8 Hz), 7.89(dd, 1H, J=6, 1.4 Hz), 7.82(dd, 2H, J=9, 2.1 Hz), 7.41(m, 1H), 7.01(d, 1H, J=8 Hz), 6.95(m, 1H).

Mass Spec (M+1)=255.9

### Ex. 39

¹H NMR (DMSO-d₆) δ12.1(s, 1H), 10.6(s, 1H), 9.3(br s, 2H), 9.1(br s, 2H), 8.0(m, 3H), 7.85(m, 2H), 7.65(br s, 1H), 7.4(br s, 1H), 6.6(m, 2H).

Mass Spec (M+1)=329.3

### Ex. 41

¹H NMR (DMSO-d₆) δ11.95(br s, 1H), 10.5(br s, 1H), 10.35(br s, 1H), 9.25(br s, 2H), 8.9(br s, 2H), 7.9(m, 5H), 6.4(m, 2H).

Mass Spec (M+1)=271.7

### Ex. 45

¹H NMR (DMSO-d₆) δ11.65(br, 1H), 10.7(s, 1H), 9.3(br s, 2H), 9.0(br s, 2H), 8.15(s, 1H), 7.95(d, 2H), 7.85(d, 2H), 7.7(d, 1H), 6.9(d, 1H)

Mass Spec (M+1)=382.1

### Ex. 49

¹H NMR (DMSO-d₆) δ12.05(br s, 1H), 11.3(br s, 1H), 10.5(s, 1H), 9.3(br s, 2H), 9.0(br s, 2H), 8.2(s, 1H), 7.9(m, 4H), 6.7(s, 1H).

### Ex. 63

¹H NMR (DMSO-d₆) δ9.0(br, 4), 8.28(d, 1H), 7.89(m, 2H), 7.78(m, 3H), 7.33(m, 1H), 7.18(m, 1H).

Mass Spec (M+1)=322.3

### Ex. 65

¹H NMR (DMSO-d₆) δ12.45(s,1H), 10.9(s,1H), 9.3(s,2H), 8.95(s,2H), 8.1(d,1H), 7.95(d, 2H), 7.55(m, 3H), 7.4(m, 3H), 7.1(t, 1H).

Mass Spec (M+1)=331.9

### Ex. 68

¹H NMR (DMSO-d₆) δ11.5(br s, 1H), 11.25(s, 1H), 10.7(s, 1H), 9.3(br s,2H), 8.9(br s, 2H), 8.15(d, 1H), 8.0(d, 2H), 7.9(d, 2H), 7.65(d, 1H), 7.1(d, 1H).

Mass Spec (M+1)=366.8

Listed in TABLE-II below are compounds wherein R⁷ is a guanidinyl group (NH—C(═NH)NH₂).

TABLE II

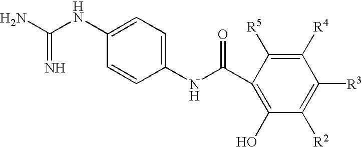

| Ex. | R² | R³ | R⁴ | R⁵ | |
|---|---|---|---|---|---|
| 150 | H | 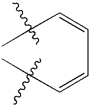 | | H | |
| 151 | H | 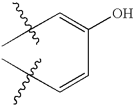 | | H | |
| 152 | Br | 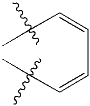 | | H | |
| 153 | Br | 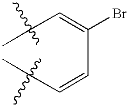 | | H | |
| 154 | H | 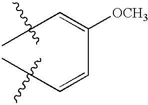 | | H | |
| 155 | Cl | 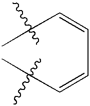 | | H | |
| 156 | H | 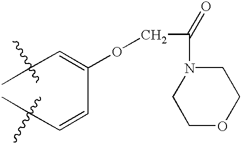 | | H | |
| 157 | I | 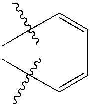 | | H | |
| 158 | H | Ph | H | H | |
| 159 | H | 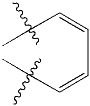 | | H | |
| 160 | H | CH₃ | H | H | |
| 161 | H | 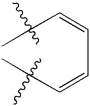 | | H | R⁶ = F |
| 162 | Br | H | CH₃ | H | |
| 163 | I | H | CH₃ | H | |
| 164 | H | OC₂H₅ | H | H | |
| 165 | I | OH | Br | H | |
| 166 | Br | H | Br | H | |
| 167 | H | 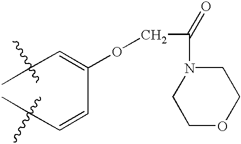 | H | H | |
| 168 | H | 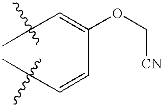 | H | H | |
| 169 | H | 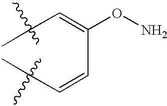 | H | H | |
| 170 | NH₂ | 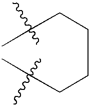 | H | H | |
| 171 | OH | 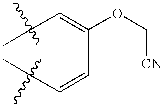 | H | H | |
| 172 | H | 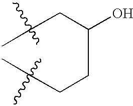 | H | H | |
| 173 | SO₃H | 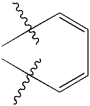 | H | H | |
| 174 | H | 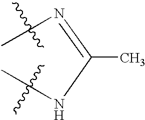 | H | H | |
| OH3 | | | | | |
| 175 | H | 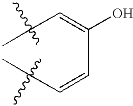 | H | H | |
| 176 | H | 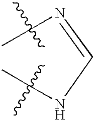 | H | H | |

Listed below is the proton NMR (¹H NMR) and Mass spectral data for compounds listed in TABLE-II.

### EX. 150

3-Acetoxy-2-naphthoic acid:

A mixture of 3-hydroxy-2-naphthoic acid (1 g, 5.3 mmol) and acetic anhydride (1 mL) was combined with con. sulfuric acid (2 drops) resulting in a solidified mixture in about 30 minutes. The solid was washed with acetic acid (15 mL) and recrystallized using a 1:1 mixture of methanol:water to yield 3-Acetoxy-2-naphthoic acid (0.68 g; 56% yield) in the form of yellow needles.

¹H-NMR (DMSO-d₆) δ8.60 (s, 1H), 8.11 (d, 1H, J=8.1 Hz), 7.95 (d, 1H, J=8.1 Hz), 7.71 (s, 1H), 7.66 (t, 1H, J=7.0 Hz), 7.58 (t, 1H, 7.5 Hz), 2.30 (s, 3H).

N-(3-hydroxy-2-naphthoyl)-4-aminophenyl guanidine hydrochloride:

A suspension/mixture of 3-acetoxy-2-naphthoic acid (2.0 g, 8.7 mmol), ethyl acetate (17 mL) and catalytic amount of DMF (0.2 mL) was combined with oxalyl chloride (1.1 mL, 13 mmol) to form a mixture. The mixture was agitated for an hour. The agitated mixture was concentrated under reduced pressure to yield 3-acetoxy-2-naphthoyl chloride as a yellowish solid. The preceding naphthoyl chloride and 4-aminophenylguanidine hydrochloride (1.94 g, 8.7 mmol) was suspended in N,N-dimethyl acetamide (DMA). This suspension was agitated for about 8 to 16 hours to form a solution. The solution was diluted with ether (150 mL) and the diluted reaction mixture was agitated vigorously for about 5 minutes forming a precipitate. The precipitate was isolated and dried to yield N-(3-acetoxy-2-naphthoyl)-4-aminophenyl guanidine.

An aqueous mixture of the preceding N-(3-acetoxy-2-naphthoyl)-4-aminophenyl guanidine hydrochloride was treated with 2 N NaOH (18 mL, 36 mmol) at a temperature of about 70° C. for about 8 hours. Conversion of the acetoxy group to a hydroxy group was confirmed by MS (CI) analysis. The reaction mixture then was acidified with 6 M HCl leading to the formation of a golden-yellow colored precipitate. This precipitate was isolated, washed with water and dried to yield N-(3-hydroxy-2-naphthoyl)-4-aminophenyl guanidine hydrochloride (2.75 g). This guanidine hydrochloride was purified by flash chromatography.

The purified N-(3-hydroxy-2-naphthoyl)-4-aminophenyl guanidine hydrochloride was dissolved in aqueous dilute NaOH. This NaOH solution was acidified to a pH of about 6-7 using 6 M HCl leading to precipitate formation. The precipitate was isolated and dried to yield N-(3-hydroxy-2-naphthoyl)-4-aminophenyl guanidine hydrochloride as a tan colored solid (1.36 g; 44% yield).

¹H NMR (DMSO-d₆) δ11.33 (3, 1H), 10.71 (s, 1H), 9.84 (s, 1H), 8.49 (s, 1H), 7.92 (d, 1H, J=8.2 Hz), 7.8 (s, 1H, J=8.5 Hz), 7.75 (d, 1H, J=8.3 Hz), 7.55-7.30 (m, 7H), 7.25 (d, 2H, J=8.6 Hz).

Mass Spec (M+1)=321.0

### Ex. 152

¹H-NMR (DMSO-d₆) δ11.1 (s, 1H), 9.8 (s, 1H), 8.7 (s, 1H), 8.1 (d, 1H), 8.0 (d, 1H), 7.8 (d, 2H), 7.7 (t, 1H), 7.5-7.3 (m, 4H), 7.2 (d, 2H).

Mass Spec (M+1)=400.7

### Ex. 155

¹H-NMR (DMSO-d₆) δ11.9 (br s, 1H), 10.9 (s, 1H), 9.7 (s, 1H), 8.6 (s, 1H), 8.0 (d, 1H), 7.9 (d, 1H), 7.7 (d, 2H), 7.6 (t, 1H), 7.5-7.3 (m, 4H), 7.2 (d, 2H).

Mass Spec (M+1)=354.8

### Ex. 157

¹H-NMR (DMSO-d₆) δ12.73 (s, 1H), 11.15 (s, 1H), 9.93 (s, 1H), 8.90 (s, 1H), 8.00 (d, 1H, J=8.55 Hz), 7.95 (d, 1H, J=8.07 Hz), 7.86 (d, 2H, J=8.71 Hz), 7.70 (t, 1H, J=7.66 Hz), 7.60-7.45 (m, 4H), 7.29 (d, 2H, J=8.65 Hz).

Mass Spec (M+1)=446.9

### Ex. 159

¹H-NMR (DMSO-d₆) δ11.30 (s, 1H), 10.75 (s, 1H), 10.00 (s, 1H), 8.47 (s, 1H), 7.93 (d, 1H, J=8.18 Hz), 7.79 (s, 1H), 7.77 (d, 1H, J=8.52 Hz), 7.65 (d, 1H, J=8.52 Hz), 7.58-7.32 (m, 7H), 7.01 (d, 1H, J=8.18 Hz).

Mass Spec (M+1)=320.9

### Ex. 160

¹H NMR (DMSO-d₆) δ11.87 (s, 1H), 10.42 (s, 1H), 9.81 (s, 1H), 7.89 (d, 1H, J=7.97 Hz), 7.78 (d, 2H, J=8 .09 Hz), 7.43 (s, 3H), 7.22 (d, 2H, J=8.56 Hz), 6.80-6.70 (m, 2H), 2.28 (s, 3H). Mass Spec (M+1)=284.9.

### Ex. 162

¹H NMR (DMSO-d₆) δ12.60 (s, 1H), 10.73 (s, 1H), 9.94 (s, 1H), 7.79-7.94 (m, 2H), 7.79 (d, 2H, 8.91), 7.65 (s, 1H), 7.50 (s, 2H), 7.27 (d, 2H, J=8.66), 2.30 (s, 3H).

Mass Spec (M+1)=364.8

### Ex. 163

¹H NMR (DMSO-d₆) δ12.83 (s, 1H), 10.71 (s, 1H), 9.89 (s, 1H), 7.98 (s, 1H), 7.84 (s, 1H), 7.78 (d, 2H, J=8.91), 7.48 (s, 2H), 7.27 (d, 2H, J=8.91), 2.29 (s, 3H).

Mass Spec (M+1)=410.8

### Ex. 164

¹H NMR (DMSO-d₆) δ12.33 (s, 1H), 10.33 (s, 1H), 9.75 (s, 1H), 7.99 (d, 1H, J=8.71), 7.78 (d, 2H, J=8.71), 7.40 (s, 2H), 7.23 (d, 2H, J=8.71), 6.55 (dd, 1H, J =8.71, 2.38), 6.49 (d, 1H, J=2.38), 4.07 (q, 2H, J=6.86 Hz), 1.43 (t, 3H, J=6.86 Hz).

Mass Spec (M+1)=314.8.

### Ex. 167

¹H-NMR (d₆-DMSO) δ (ppm): 11.07 (s, 1H), 11.01 (br s, 1H), 10.73 (s, 1H), 8.73 (s, 1H), 8.32 (s, 1H), 8.23-8.08 (m, 4H), 7.69 (d, 1H, J=8.8 Hz), 7.29 (s, 2H), 7.20 (d, 1H, J=8.8 Hz), 7.10 (d, 1H, J=8.8 Hz), 4.90 (s, 2H), 3.60-3.44 (m, 8H).

MS (ES) calc. 464.5, found 465.2 (MH+).

This compound was prepared by the following process:

3,7-Dihydroxy-naphthalene-2-carboxylic acid benzyl ester

A mixture of 3,7-dihydroxy-naphthalene-2-carboxylic acid (10.0 g, 49 mmol) and NaHCO₃ (10.3 g, 123 mmol) in 70 mL of N,N-dimethylformamide was agitated for approximately 12 hours at ambient temperature and at about 70° C. for an additional 4 hours. The mixture was cooled to about 40° C. and then combined with benzyl bromide (7 mL, 59 mmol). The resulting mixture was agitated at about 70° C. for about 12 hours. The preceding agitated reaction mixture was concentrated under reduced pressure, diluted with AcOEt and the diluted mixture was sequentially washed with satd. NaHCO₃, said NaCl, 0.5 M HCl, and satd. NaCl, dried (Na₂SO₄) and concentrated under reduced pressure to afford a brown oil. The brown oil was diluted with hexanes to form a precipitate which was isolated to afford the benzyl ester as a golden powder (11.65 g, 81%). ¹H-NMR (d₆-DMSO) δ (ppm): 9.95 (s, 1H), 9.62 (s, 1H), 8.23 (s, 1H), 7.60 (d, 1H, J=8.8 Hz), 7.50 (d, 2H, J=7.3 Hz), 7.43-7.34 (m, 3H), 7.22 (s, 1H), 7.13-7.09 (m, 2H), 5.40 (s, 2H).

3-Hydroxy-7-(2-morpholin-4-yl-2-oxo-ethoxy)-naphthalene-2-carboxylic acid benzyl ester A mixture of morpholine (2.16 mL, 25 mmol) and anhydrous ether (30 mL) was cooled (−10° C.) and treated drop wise with a solution of bromoacetyl bromide (5.0 g, 25 mmol) in ether (20 mL). Triethyl amine (3.5 mL, 25 mmol) then was added drop wise to the reaction mixture to form a cream colored reaction mixture. The creamy reaction mixture was agitated at about 20° C. for about 6 hours. The reaction solids were isolated and rinsed with ether. The combined ether fractions were concentrated under reduced pressure to afford N-(2-bromoacetyl)-morpholine (3.37 g) as a reddish oil, which was used without further purification.

A solution of the N-(2-bromoacetyl)-morpholine (2.09 g, 10 mmol) in acetone (5 mL) was introduced in a drop wise manner into a mixture of 3,7-Dihydroxy-naphthalene-2-carboxylic acid benzyl ester (2.69 g, 9.1 mmol) and K₂CO₃ (1.39 g, 10.1 mmol) in 15 mL of acetone. The combined mixture was heated to reflux for about 12 hours, at which time another 0.2 g (1.0 mmol) of N-(2-bromoacetyl)-morpholine and 0.24 g of K2CO3 (1.7 mmol) were added and the heating continued for an additional 3 hours. The mixture was cooled to ambient temperature, diluted with AcOEt, washed with water and said. NaCl, dried (Na₂SO₄) and concentrated under reduced pressure to yield an oily residue. The oily residue was purified by chromatography (silica) using a gradient elution employing 50 to 80% AcOEt in hexanes. The title compound was obtained as a yellow foam (1.06 g, 28%). ¹H-NMR (d₆-DMSO) δ (ppm): 10.08 (s, 1H), 8.31 (s, 1H), 7.687 (d, 1H, J=9.2 Hz), 7.50 (d, 2H, J=7.0 Hz), 7.44-7.37 (m, 3H), 7.29 (s, 1H), 7.23 (d, 2H, J=8.8 Hz), 5.41 (s, 2H), 4.85 (s, 2H), 3.59-3.44 (m, 8H).

3-Acetoxy-7-(2-morpholin-4-yl-2-oxo-ethoxy)-naphthalene-2-carboxylic acid chloride

3-Hydroxy-7-(2-morpholin-4-yl-2-oxo-ethoxy)-naphthalene-2-carboxylic acid benzyl ester (1.06 g, 2.5 mmol) was hydrogenated at atmospheric pressure in 10 mL of tetrahydrofuran over 10% Pd—C (wet) for 2 hours. The catalyst was removed by filtration, and solvent was removed under reduced powder to yield the carboxylic acid as a yellow solid (0.77 g, 93%) was used without further purification.

3-Hydroxy-7-(2-morpholin-4-yl-2-oxo-ethoxy)-naphthalene-2-carboxylic acid (from above) was moistened with 3 mL of acetic anhydride and 2 drops of conc. H₂SO₄. The resulting heterogeneous mixture was agitated for 20 min, and the undissolved solids were dissolved by adding 1 mL glacial AcOH. The resulting reaction mixture was concentrated under reduced pressure, the concentrated reaction mixture was diluted with AcOEt (250 mL), dried (Na₂SO₄) and concentrated under reduced pressure to afford the 3-Acetoxy-7-(2-morpholin-4-yl-2-oxo-ethoxy)-naphthalene-2-carboxylic acid as a pale yellow oil, which was taken directly onto the next step. Oxalyl chloride (0.25 mL, 2.8 mmol) was added drop wise to a mixture of the 3-Acetoxy-7-(2-morpholin-4-yl-2-oxo-ethoxy)-naphthalene-2-carboxylic acid (from above), 5 mL of 1,4-dioxane and 0.1 mL of N,N-dimethylformamide. The resulting solution was agitated for about 1 hour. The agitated reaction mixture was concentrated under reduced pressure to yield the acyl chloride which was used without further purification coupling with the appropriate aniline derivative to yield the compound of Example 167.

TABLE-III below lists compounds wherein R⁷ is a guanidinyl group (NH—C(═NH)NH₂) and X₁ represents a nitrogen atom.

TABLE III

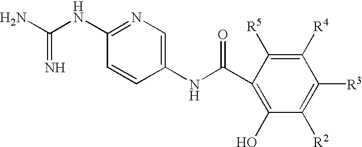

| Ex. | R² | R³ | R⁴ | R⁵ |
|---|---|---|---|---|
| 200 | H | H | H | H |
| 201 | H | 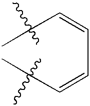 | H | |
| 202 | H | OC₂H₅ | H | H |
| 203 | H | H | CH₃ | H |
| 204 | H | H | H | OH |
| 205 | I | H | CH₃ | H |
| 206 | H | CH₃ | H | H |
| 207 | H | 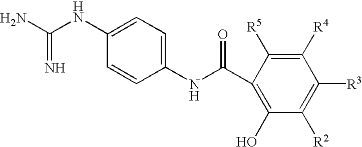 | H | |
| 208 | H | 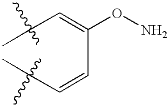 | H | |
| 209 | H | 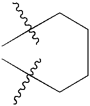 | H | |
| 210 | H | 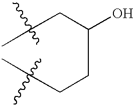 | H | |
| 211 | NH₂ | 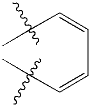 | H | |
| 212 | OH | 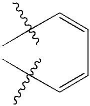 | H | |
| 213 | H | 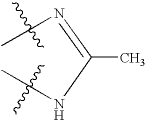 | H | |
| 214 | SO₃H | 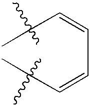 | H | |
| 215 | H | 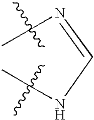 | H | |
| 216 | H | 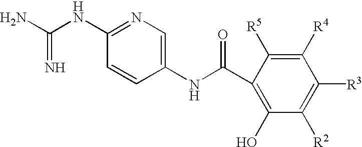 | H | |

Listed below is the proton NMR (¹H NMR) and Mass spectral data for compounds listed in TABLE-II.

### Ex. 200

¹H NMR (DMSO-d₆) δ11.67 (s, 1H), 11.26 (s, 1H), 10.59 (s, 1H), 8.71 (d, 1H, J=2.48), 8.21-8.17 (m, 3H), 7.95 (dd, 1H, J=1.24, 8.17), 7.45 (td, 1H, J=1.73, 8.91, 8.42), 7.11 (d, 1H, J=8.91), 7.00 (d, 1H, J=8.91), 6.96 (d, 1H, J=7.43).

Mass Spec (M+1)=271.8

Ex. 201: 3-hydroxy-naphthalene-2-carboxylic acid (6-guanidino-pyridin-3-yl)-amide

This compound was prepared by reacting 3-acetoxy-naphthalene-2-carboxylic acid chloride (alternatively named as acetic acid 3-chlorocarbonyl-naphthalen-2-yl ester) with N-(5-Amino-pyridin-2-yl)-guanidine hydrochloride. N-(5-Amino-pyridin-2-yl)-guanidine hydrochloride was prepared as described below.

N-(5-Amino-pyridin-2-yl)-guanidine hydrochloride

The first step comprised synthesis of N-(5-nitro-pyridin-2-yl)-guanidine using the procedure of Carbon and Tabata described in J. Org. Chem (1962) 2504-7.

¹H NMR (DMSO-d₆) δ12.23 (s, 1H), 9.12 (d, 1H, J=2.97 Hz), 8.62 (dd, 1H, J=2.97, 8.91 Hz), 8.49 (s, 2H), 7.26 (d, 1H, 8.91 Hz).

The second step comprised synthesizing N-(5-amino-pyridin-2-yl)-guanidine hydrochloride by preparing a mixture of N-(5-nitro-pyridin-2-yl)-guanidine hydrochloride (15.82 g; 73 mmol) and 10% Pd/C (100 mg) and methanol (1 L). This mixture then was agitated in an atmosphere of hydrogen for 2 hours. The agitated mixture was filtered and the filtrate concentrated to yield N-(5-amino-pyridin-2-yl)-guanidine hydrochloride (13.4 g) as a yellow solid.

¹H NMR (DMSO-d₆) δ10.88 (s, 1H), 8.01 (s, 2H), 7.65 (d, 1H, J=2.72 Hz), 7.09 (dd, 1H, J=2.72, 8.66 Hz), 6.80 (d, 1H, J=8.66 Hz), 5.29 (d, 2H, J=4.70 Hz).

3-acetoxy-naphthalene-2-carboxylic acid chloride (alternatively named as acetic acid 3-chlorocarbonyl-naphthalen-2-yl ester)

The acid chloride, above, was prepared by treating a mixture of 2-acetoxy-3-naphthoic acid (5 g, 22 mmol), EtOAc (80 ml) and DMF (3 drops) with oxalyl chloride (2.8 ml, 1.5 eq). The resulting reaction mixture was agitated for 0.5 h and the agitated mixture was concentrated in vacuo to a yield 3-acetoxy-naphthalene-2-carboxylic acid chloride as a yellow solid.

EX.: 201 3-hydroxy-naphthalene-2-carboxylic acid (6-guanidino-pyridin-3-yl)-amide

The above acid chloride (1 eq.) was mixed with DMA (20 ml) and N-(5-amino-pyridin-2-yl)-guanidine hydrochloride (5.33 g, 1.3 eq) and the resulting mixture was agitated for 8-16 hours under an atmosphere of nitrogen. The agitated reaction mixture then was mixed with conc. ammonium hydroxide (150 ml) to form a yellow precipitate. The precipitate was isolated, dried and mixed with 1 M HCl. The mixture was agitated for 2 h, the resulting solids were isolated and dried to yield 3-hydroxy-naphthalene-2-carboxylic acid (6-guanidino-pyridin-3-yl)-amide (6.4 g, 78%) as a pale yellow solid.

¹H NMR (DMSO-d₆) δ11.24 (s, 1H), 11.19 (s, 1H), 10.77 (s, 1H), 8.77 (d, 1H, J=2.23), 8.49 (s, 1H), 8.24 (dd, 1H, J=2.48, 8.91), 8.21 (s, 1H), 7.93 (d, 1H, J=7.92), 7.77 (d, 1H. J=8.42), 7.52 (t, J=6.93, 7.18), 7.39-7.34 (m, 2H), 7.13 (d, 1H, J=8.91).

Mass Spec (M+1)=321.8

### Ex. 202

¹H NMR (DMSO-d₆) δ8.60 (s, 1H), 8.06 (d, 1H, J=8.66), 7.73 (d, 1H, J=8.66), 6.97 (d, 1H, J=8.91), 6.14-6.10 (m, 2H), 3.95 (q, 2H, J=6.68), 1.28 (t, 3H, J=6.68).

Mass Spec (M+1)=315.8

### Ex. 203

¹H NMR (DMSO-d₆) δ8.57 (s, 1H), 8.02 (d, 1H, J=8.91), 7.65 (s, 1H), 7.07 (d, 1H, J=8.17), 6.92 (d, 1H, J=8.91), 6.69 (d, 1H, J=8.42), 2.20 (s, 3H).

Mass Spec (M+1)=285.8

### Ex. 205

¹H NMR (DMSO-d₆) δ11.37 (s, 1H), 10.86 (s, 1H), 8.67 (d, 1H, J=2.23), 8.23-8.16 (m, 3H), 7.98 (s, 1H), 7.84 (d, 1H, J=1.73), 7.13 (d, 1H, J=8.91), 2.29 (s, 3H).

Mass Spec =411.7

### Ex. 206

¹H NMR (DMSO-d₆) δ8.69 (d, 1H, J=2.72), 8.17 (dd, 1H, J=2.72, 8.91), 7.87 (d, 1H, J=7.92), 7.09 (d, 1H, J=8.66), 6.79 (s, 1H), 6.76 (d, 1H, J=8.42), 2.29 (s, 3H).

Mass Spec (M+1)=285.9

Utility

Proteases play a significant role in the progression of Cancer. Compounds of the present invention are useful as protease inhibitors. Their inhibitory activity includes inhibition of urokinase (uPA) which has been postulated to have therapeutic value in treating cancers such as lung cancer, breast cancer, pancreatic cancer, colon cancer, ovarian cancer, bone cancer and the like.

The compounds of the present invention are also useful as anticoagulants for the treatment or prevention of thromboembolic disorders in mammals. The term “thromboembolic disorders” as used herein includes arterial or venous cardiovascular or cerebrovascular thromboembolic disorders, including, for example unstable angina, first or recurrent ischemic attack, stroke, atherosclerosis, venous thrombosis, deep vein thrombosis, thrombophlebitis, arterial embolism, kidney embolisms, and pulmonary embolisms. The anticoagulant effect of compounds of the present invention is believed to be due to the inhibition of Factor Xa (FXa), Factor VIIa (FVIIa), and thrombin.

Some of the compounds of the present invention show selectivity between uPA and FXa, with respect to their inhibitory properties. The effectiveness of compounds of the present invention as inhibitors of Urokinase and Factor Xa is determined by using synthetic substrates and purified Urokinase and purified human Factor Xa respectively.

The rates of hydrolysis by the chromogenic substrates were measured both in the absence and presence of compounds of the present invention. Hydrolysis of the substrates result in the release of a chromogenic moiety, which is monitored spectrophotometrically by measuring the increase in absorbance at 405 nano meter (nm). A decrease in the rate of absorbance change at 405 nm in the presence of a inhibitor is indicative of enzyme inhibition. The results of this assay are expressed as the inhibitory constant, Ki app. Factor Xa determinations were made in 50 mM Tris buffer, pH 7.5, containing 1 M NaCl, 5 mM CaCl₂, 0.05% Tween-20, and 1.5 mM EDTA. Values of Ki app. were determined by allowing 2-3 nM human Factor Xa (Haematologic Technologies, Vt., USA) to react with the substrate (1 mM) in the presence of an inhibitor. Hydrolysis of the chromogenic substrate is followed spectrophotometrically at 405 nm for five minutes. The enzyme assay routinely yielded linear progression curves under these conditions. Initial velocity measurements calculated from the progress curves by a kinetic analysis program (Batch Ki; Peter Kuzmic, BioKin, Ltd., Madison, Wis.) were used to determine Ki app.

Urokinase inhibition determinations were made in 50 mM Tris (pH 7.5), 150 mM NaCl, 0.05% Tween-20, 0.002% antifoam, and 1 mM EDTA. human Urokinase (from American Diagnostica, Conn., USA). Values of Ki app. were determined by allowing 20 nM human Urokinase to react with the Pefachrome substrate (0.3 mM, Centerchem, Conn., USA) in the presence of an inhibitor. Hydrolysis of the chromogenic substrate is followed spectrophotometrically at 405 nm for five minutes. The enzyme assay routinely yielded linear progression curves under these conditions. Initial velocity measurements calculated from the progress curves by a kinetic analysis program (Batch Ki; Peter Kuzmic, BioKin, Ltd., Madison, Wis.) were used to determine Ki app.

Table IV lists inhibition constants (Ki app.) for representative compounds of the present invention. These values are for uPA and FXa.

TABLE IV

| | uPA | FXa |
|---|---|---|
| Ex. | Ki μM | Ki μM |
| 1 | 0.16 | 0.88 |
| 5 | 0.29 | 0.84 |
| 24 | 2.9 | 34 |
| 201 | 0.326 | 130 |
| 205 | 5.5 | 290 |

Definitions

The compounds of the present invention may have asymmetric centers. Compounds of the present invention containing an asymmetrically substituted atom may be isolated in optically active or racemic forms. It is well known in the art how to prepare optically active forms, such as by resolution of materials. Many geometric isomers of olefins, C═N double bonds, and the like can be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure (representing a compound of Formula I) are intended, unless the specific stereochemistry or isomeric form is specifically indicated.

As used herein, the following terms and abbreviations have the following meaning, unless indicated otherwise.

The term “prodrug” is intended to represent covalently bonded carriers which are capable of releasing the active ingredient of Formula I, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic or a similar group is modified. “Pharmaceutically acceptable salts” is as understood by one skilled in the art. Thus a pharmaceutically acceptable salt includes acid or base salts of compounds of Formula I. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Optional” or “optionally” means that the subsequently described event or circumstance may or may not occur, and that the description includes instances where the event or circumstance occurs and instances in which it does not. For example, the phrase “optionally is substituted with one to three substituents” means that the group referred to may or may not be substituted in order to fall within the scope of the invention. Thus the term “optionally substituted” is intended to mean that any one or more hydrogens on a designated atom can be replaced with a selection from the indicated group, provided that the designated atom's normal valence is not exceeded, and that the substitution results in a stable compound. When the substituent is keto (═O) then 2 hydrogens on the atom are replaced. There are one to three “optional substituents”, unless otherwise indicated, and these substituents are independently selected from a group consisting of H; N(R¹⁰)₂; NO₂; halogen; aryl; O—C₅₋₁₀cyclo alkyl substituted with R¹⁰; guanidino; urea; thio urea; amidino; para or meta phenoxy; piperidin-4-yloxy; 4-amino-cyclohexyloxy; 1-(1-Imino-ethyl)-piperidin-4-yloxy; 1-(1-Imino-ethyl)-pyrrolidin-3-yloxy; 2-Amino-3-methyl-butyryl; 4-Acetimidoylamino-cyclohexyloxy; 1-(1-Imino-ethyl)-pyrrolidin-2-ylmethoxy; 2-(2-Hydroxycarbonimidoyl-pyridin-3-yloxy)-ethoxy; 3,4-Dicyano-phenoxy; SC₁₋₄alkyl, S-aryl, O—C₁₋₄alkyl, COOR¹⁰, OR¹⁰, C(O)-pyrrolidine; C(O)CH(NH₂)CH₂OH; C(O)CH(NH₂)CH₂Ph; C(O)CH(NH₂)CH₂COOH; O-pyrrolidine; C(O)—(CH₂)₁₋₃-imidazole; SO₂-N(alkyl)₂; C(═N)—C₃; O-piperidine; 2-aminothiazol-5-ylmethoxy; O-CH₂—COOH; pyrrolidine-2-ylmethoxy; 2,4,6-triamino pyrimidin-5-ylmethoxy; NH—SO₂-alkyl; NHC₁—C₄alkyl; N(C₁—C₄)₂alkyl; CF₃; C₂₋₁₀alkenyl and C₁₋₁₀alkyl.

The term “alkyl”, as used herein, is intended to include branched and straight chain saturated aliphatic hydrocarbon groups having from 1 to 14 or the specified number of carbon atoms, illustrative examples of which include, but are not limited to, methyl, ethyl, n-propyl, i-propyl, n-butyl, i-butyl, sec-butyl, t-butyl, n-pentyl, and n-hexyl. “Alkenyl” is intended to include a branched or straight chain hydrocarbon group having one or more unsaturated carbon-carbon bonds which may occur in any stable point along the chain, such as ethenyl, propenyl, and the like. The term “alkelene” represents an alkyl group, as defined above, except that it has at least one center of unsaturation, i.e., a double bond. Illustrative examples are butene, propene, and pentene. The term “cycloalkyl”, “cycloalkyl ring”, “cycloalkyl radical” or “cyclic hydrocarbon” indicates a saturated or partially unsaturated three to fourteen carbon monocyclic or bicyclic hydrocarbon moiety which is optionally substituted with an alkyl group. Illustrative examples include cyclo propyl, cyclo hexyl, cyclo pentyl, and cyclo butyl. The term “alkoxy” as used herein represents —OC₁₋₆alkyl.

The terms “Ar” and “aryl”, as used herein, are intended to represent a stable substituted or unsubstituted (collectively also referred to as ‘optionally substituted’) six to fourteen membered mono-, bi- or tri-cyclic hydrocarbon radical comprising carbon and hydrogen atoms. Illustrative examples are phenyl (Ph), naphthyl, anthracyl groups, and piperanyl. It is also intended that the terms “carbocycle” and “carbocyclic” include “Ar”, “aryl” as well as “cyclo alkyl” groups, which are defined above. “Halogen” or “halo”, as used herein, represents Cl, Br, F or I.

The term “heteroaryl” is intended to represent a stable 5 to 10 membered aryl group (“aryl” as defined above), wherein one or more of the carbon atoms is replaced by a hetero atom selected from N, O, and S. The hetero atoms can exist in their chemically allowed oxidation states. Thus a Sulfur (S) atom can exist as a sulfide, sulfoxide, or sulfone. Preferred heteroaryl groups are six membered ring systems comprising not more than 2 hetero atoms. Illustrative examples of preferred heteroaryl groups are thienyl, N-substituted succinimide, 3-(alkyl amino)-5,5-dialkyl-2-cyclohexen-1-one, methyl pyridyl, alkyl theophylline, furyl, pyrrolyl, indolyl, pyrimidinyl, isoxazolyl, purinyl, imidazolyl, pyridyl, pyrazolyl, quinolyl, and pyrazinyl. The term “heterocycloalkyl” means a stable cyclo alkyl group containing from 5 to 14 carbon atoms wherein one or more of the carbon atoms is replaced by a hetero atom chosen from N, O and S. The hetero atoms can exist in their chemically allowed oxidation states. Thus Sulfur (S) can exist as a sulfide, sulfoxide, or sulfone. The heterocycloalkyl group can be completely saturated or partially unsaturated. Illustrative examples are piperidine, 1,4-dioxane, and morpholine.

As used herein the terms “heterocyclyl”, “heterocyclic” and/or “het” are intended to represent a stable 5- to 7- membered monocyclic or 7- to 10- membered bicyclic heterocyclic ring which is saturated, partially unsaturated, or unsaturated (aromatic), which consists of carbon atoms and from one to 4 hetero atoms independently selected from a group consisting of N, O and S. The nitrogen and the sulfur hetero atoms can exist in their respective oxidized states. The heterocyclic ring may be attached to its pendent group at any hetero atom or carbon atom which results in a stable structure. The heterocyclic rings described herein may be substituted on a carbon or a nitrogen atom if the resulting compound is stable. The nitrogen in the heterocycle can exist in its quaternized form. It is preferred that when the total number of hetero atoms in the heterocycle exceeds 1, then the hetero atoms are not adjacent to one another. It is understood that the terms “heterocyclyl”, “heterocyclic”, and “het” include the terms “heteroaryl”, “heterocycloalkyl” and “bicyclic heterocyclic ring structure” as described above.

Preferred “heterocyclyl”, “heterocyclic” and/or “het” groups are selected from 1-(2-Hydroxymethyl-pyrrolidin-1-yl)-2,3-dimethyl-butan-1-one, 3-Pyridin-2-yl-propan-1-ol, N-(2,3-Dimethoxy-benzyl)-2-hydroxy-acetamide, 1-Methyl-2-m-tolyl-1H-benzoimidazole-5-carboxamidine, 2-Methyl-3,4,6,7-tetrahydro-imidazo[4,5-c]pyridine-5-carboxamidine, 2-Amino-3-hydroxy-1-(2-methyl-3,4,6,7-tetrahydro-imidazo[4,5-c]pyridin-5-yl)-propan-1-one, 2-Amino-1-(2-methyl-3,4,6,7-tetrahydro-imidazo[4,5-c]pyridin-5-yl)-ethane, 2-Methyl-4,5,6,7-tetrahydro-3H-imidazo[4,5-c]pyridine, N-o-Tolyl-methanesulfonamide, 2-Methyl-benzothiazole, 3-Amino-1-(2-hydroxymethyl-pyrrolidin-1-yl)-propan-1-one, 2-Hydroxy-1-(2-hydroxymethyl-pyrrolidin-1-yl)-ethanone, 2-(2-Hydroxy-ethyl)-indan-1,3-dione, 5-Fluoro-2-methyl-1H-benzoimidazole, 2-Methyl-1H-imidazo[4,5-c]pyridine, 2-Hydroxy-N-(2-morpholin-4-yl-ethyl)-acetamide, 2-Methyl-1H-imidazo [4,5-b]pyridine, 2-Amino-1-(3-methyl-piperidin-1-yl)-ethanone, 2-Methyl-1H-benzoimidazol-4-ol, 2-Pyridin-2-yl-ethanol, N-(3-Hydroxy-propyl)-2-phenyl-acetamide, N-(3-Hydroxy-propyl)-3-phenyl-propionamide, N-(3-Hydroxy-propyl)-benzamide, N-(2-Hydroxy-ethyl)-2-phenyl-acetamide, (4-Hydroxy-butyl)-carbamic acid tert-butyl ester, (2-Hydroxy-ethyl)-carbamic acid benzyl ester, (4-Hydroxy-piperidin-1-yl)-phenyl-methanone, 4-Bromo-2-methoxy-benzylamine, 3-Methoxy-5-trifluoromethyl-benzylamine, N-(3,5-Dimethoxy-benzyl)-acetamide, 2-Methyl-1H-benzoimidazole-5-carboxamidine, and 2-Hydroxy-N-naphthalen-1-yl-acetamide.

The following structural representations further illustrate the term “het”:

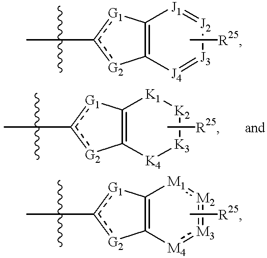

wherein G₁ and G₂ independently at each occurrence represent S(O)₀₋₂, NH, N—R²⁴, O, CR¹⁰, or CHR¹⁰; J₁, J₂, J₃, and J₄ independently represent CR¹⁰ or N, wherein at least two of J₁, J₂, J₃, and J₄ represent CH; K₁, K₂, K₃ and K₄ independently represent —NHR, —NHR₂₄, —CHR¹⁰, —CH—C(═NH)—NH₂, or N—C(═NH)—NH₂ wherein at least two of K₁, K₂, K₃ and K₄ represent CH₂ ; M₁, M₂, M₃ and M₄ independently represent —NHR¹⁰, —NHR²⁴, —CHR¹⁰, —CH—C(═NH)—NH₂, or N—C(═NH)—NH₂, wherein at least two of M₁, M₂, M₃ and M₄ represent CH or CH₂; and R²⁵ represents H, halogen, —C₁₋₆alkyl, —NO₂, NHR¹⁰, NH—SO₂—R¹⁰, —OH, C₁₋₆alkoxy, amidino, guanidino, —COOR¹⁰, or —CONHR¹⁰. The variables R¹⁰ and R²⁴ are as defined earlier. The dashed lines indicate optional unsaturation without violating the valency rules.

The term “basic group” as used under R⁷ and R⁸, defined earlier, is intended to represent amidino, guanidino, —C(═NH)N(R¹⁰)₂, 2-imidazoline, —N-amidinomorpholine, N-amnidino piperidine, 4-hydroxy-N-amidino piperidine, N-amidino pyrrolidine, tetrahydro pyrimidine, and thiazolidin-3-yl-methylideneamine. The compounds of the present invention were named using the “Autonom”, a Beilstein Commander 2.1 Application, distributed by Beilstein.

The term “acylatable group” as used herein represents a group which is capable of reacting with an acylating group to form an amido group. Illustrative examples of acylatable groups are primary or secondary amino, guanidino and amidino.

The term “acylating agent” as used herein represents a chemical agent which is capable of reacting with an acylatable group to form an amido group. Illustrative examples of an acylating agent are acid chloride and N-methylpyrrolidone.

The term “acetamide” as used herein represents a reagent that comprises an acetamide group. Illustrative examples of an acetamide are alkyl acetamide, dialkyl acetamide, dimethyl acetamide, dialkyl propionamide, and diethyl acetamide. The acetamide functions as a solvent and a base in the process of the present invention.

The term “natural amino acid”, as used herein is intended to represent the twenty naturally occurring amino acids in their ‘L’ form, which are some times also referred as ‘common amino acids’, a list of which can be found in Biochemistry, Harper & Row Publishers, Inc. (1983). The term “unnatural amino acid”, as used herein, is intended to represent the ‘D’ form of the twenty naturally occurring amino acids described above. It is further understood that the term unnatural amino acid includes homologues of the natural amino acids, and synthetically modified form of the natural amino acids. The synthetically modified forms include amino acids having alkylene chains shortened or lengthened by up to two carbon atoms, amino acids comprising optionally substituted aryl groups, and amino acids comprised halogenated groups, preferably halogenated alkyl and aryl groups.

The term “natural amino acid side chain” is intended to represent a natural amino acid (“natural amino acid” as defined above) wherein a keto (C═O) group replaces the carboxylic acid group in the amino acid. Thus, for example, an alanine side chain is C(═O)—CH(NH₂)—CH₃; a valine side chain is C(═O)—CH(NH₂)—CH(CH₃)₂; and a cysteine side chain is C(═O)—CH(NH₂)—CH₂—SH. The term “unnatural amino acid side chain” is intended to represent an unnatural amino acid (“unnatural amino acid” as defined above) wherein a keto (C═O) group replaces the carboxylic acid group forming unnatural amino acid side chains similar to ones illustrated under the definition of “natural amino acid side chain” above.

It thus follows that a “N-natural amino acid side chain” substituent and “N-unnatural amino acid side chain” substituent, which can represent Q, Q¹, Q², Q³, L¹, L², L³ and L⁴, is a group wherein the nitrogen atom (N) is the annular ring atom substituted with a natural or unnatural amino acid side chain (natural or unnatural amino acid side chain is a defined above). The point of attachment between the nitrogen atom and the natural or unnatural amino acid side chain is at the keto (C═O) group of the respective amino acids. Thus a N-natural amino acid, i.e., N-cysteine, is N—C(═O)—CH(NH₂)—CH₂—SH.

1. A compound of Formula I: 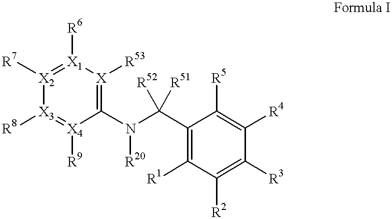
its prodrug form or pharmaceutically acceptable salts thereof, wherein:
R¹ represents OH, COOH, COO—C₁₋₄alkyl, CH₂OR¹⁰, SO₂—OH, O—SO₂—OH, O—SO₂-OC₁₋₄alkyl, OP(O)(OH)₂, or OPO₃C₁₋₄alkyl;
R², R³, R⁴, and R⁵ independently at each occurrence represent H, SH, OR¹⁰, halogen, COOR¹⁰, CONR¹¹R¹², optionally substituted aryl, optionally substituted heterocyclyl, C₄₋₁₄cycloalkyl—C₁₋₄alkyl, C₁₋₄alkyl aryl, optionally substituted C₁₋₁₄ straight chain, branched or cyclo alkyl, NR¹⁰R²⁴, (CH₂)₁₋₄—NR³³R³⁴, (CH₂)₁₋₄—COOR³³, O—(CH₂)₁₋₃—CO—het, O—(CH₂)₁₋₂—NH—CO—aryl, O—(CH₂)₀₋₂—NR¹⁰—CO—NR¹⁰OR³³, O—(CH₂)₀₋₂—C(O)—NR³³R³⁴, O—(CH₂)₁₋₄—COOR¹⁰, O—(CH₂)₁₋₃—het—R³², O-optionally substituted cycloalkyl, O—(CH₂)₁₋₄—NR¹⁰—COO—t-butyl, O—(CH₂)₁₋₄—NR¹⁰R³³, O—(CH₂)₁₋₄—NR¹⁰—C(O)—C₀₋₃-alkyl-optionally substituted aryl, O—(CH₂)₀₋₆-optionally substituted aryl, (CH₂)₁₋₄—NH—C(O)O—(CH₂)₁₋₄—PhR¹³R¹⁴, NO₂, O—(CH₂)₀₋₄—C(O)—NH-tetrahydro carboline, SO₃H, CH(OH)COOR₁₀, NR¹⁰R²⁸, O—(CH₂)₁₋₃-optionally substituted het, CH₂COOCH₃, CH═CH—COOCH₃, 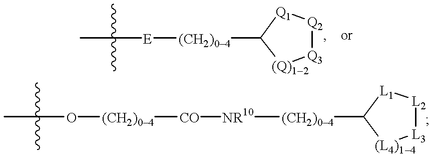
alternatively R² and R³, R³ and R⁴, or R⁴ and R⁵ taken together form 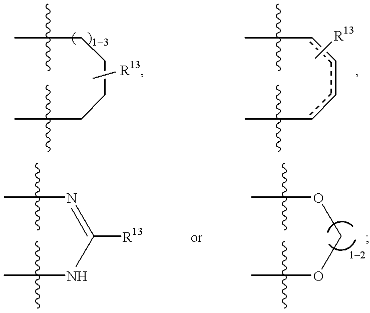
R⁶, R⁹ and R⁵³ independently at each occurrence represents H, halogen, cyano, C₁₋₄alkyl, C₁₋₄halogenated alkyl, NO₂, O-aryl or OR¹¹; alternatively R⁶ and R⁵³ taken together form 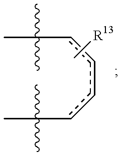
R⁷ and R⁸ independently at each occurrence represent OH, CF₃, H, COOH, NO₂, C₁₋₄alkyl, OC₁₋₄alkyl, or O-aryl, halogen, cyano, or a basic group selected from guanidino, NH(CH═NH)NH₂, C(═NH)N(R¹⁰)₂, C(═NH)—NH—NH₂, C(═O)N(R¹⁰)₂, 2-imidazoline, N-amidinomorpholine, N-amidino piperidine, 4-hydroxy-N-amidino piperidine, N-amidino pyrrolidine, tetrahydro pyrimidine, C(O)CH₂NH₂, C(O)NHCH₂CN, NHCH₂CN, and thiazolidin-3-yl-methylideneamine; with the proviso that only one of R⁷ and R⁸ represent a basic group;
R¹⁰ independently at each occurrence represents H, (CH₂)₀₋₂-aryl, C₁₋₄halo alkyl, or C₁₋₄straight chain, branched or cyclo alkyl, and alternatively, when one atom is substituted with two R¹⁰ groups, the atom along with the R¹⁰ groups can form a five to 10 membered ring structure;
X¹, X₂, X₃ and X⁴ independently at each occurrence represent a carbon or a nitrogen atom;
R¹¹ and R¹² independently at each occurrence represent H or C₁₋₄alkyl; R¹³ represents H, OH, OC₁₋₄alkyl, OAr, OC₅₋₁₀cycloalkyl, OCH₂CN, O(CH₂)₁₋₂NH₂, OCH₂COOH, OCH₂COO—C₁₋₄alkyl or 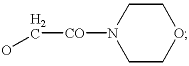
R²⁰ represents H or OH;
R²⁴ represents R¹⁰, (CH₂)₁₋₄-optionally substituted aryl, (CH₂)₀₋₄OR¹⁰, CO—(CH₂)₁₋₂—N(R¹⁰)₂, CO(CH₂)₁₋₄—OR¹⁰, (CH₂)₁₋₄—COOR¹⁰, (CH₂)₀₋₄-N(R¹⁰)₂, SO₂R¹⁰, COR¹⁰, CON(R¹⁰)₂, (CH₂)₀₋₄-aryl—COOR¹⁰, (CH₂)₀₋₄-aryl—N(R¹⁰)₂, or (CH₂)₁₋₄—het-aryl;
R²⁸ represents (CH₂)₁₋₂—Ph—O—(CH₂)₀₋₂—het—R³⁰, C(O)—het, CH₂—Ph—CH₂—het—(R³⁰)₁₋₃;
(CH₂)₁₋₄-cyclohexyl-R³¹, CH₂—Ph—O—Ph—(R³⁰)₁₋₂, CH₂—(CH₂OH)—het—R³⁰, CH₂—Ph—O—cycloalkyl-R³¹, CH₂—het—C(O)—CH₂—het—R³⁰, or CH₂—Ph—O—(CH₂)—O—het—R³⁰;
R³⁰ represents SO₂N(R¹⁰)₂, H, NHOH, amidino, or C(═NH)CH₃;
R³¹ represents R³⁰, amino-amidino, NH—C(═NH)CH₃ or R¹⁰;
R³² represents H, C(O)—CH₂—NH₂, or C(O)—CH(CH(CH₃)₂)—NH₂;
R³³ and R³⁴ independently at each occurrence represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄-N(R¹⁰)₂, (CH₂)₁₋₄—OH, (CH₂)₁₋₄—SO₂—N(R¹⁰)₂;
alternatively, R³³ and R³⁴ along with the nitrogen atom that they are attached to forms a 4 to 14 atom ring structure selected from tetrahydro-1H-carboline; 6,7-Dialkoxyoxy-2-substituted 1,2,3,4-tetrahydro-isoquinoline, 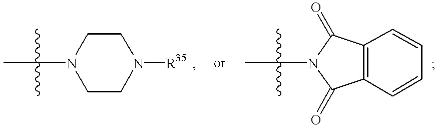
R³⁵ represents R¹⁰, SO₂—R¹⁰, COR¹⁰, or CONHR¹⁰;
E represents a bond, S(O)₀₋₂, O or NR¹⁰;
Q, Q¹, Q², Q³, L¹, L², L³ and L⁴ independently at each occurrence represent N-natural or unnatural amino acid side chain, CHR¹⁰, O, NH, S(O)₀₋₂, N—C(O)—NHR¹⁰, SO₂—N(R¹⁰)₂, N—C(O)—NH—(CH₂)₁₋₄—R²⁶, NR¹⁰, N-heteroaryl, N—C(═NH)—NHR¹⁰, or N—C(═NH)C₁₋₄alkyl;
R²⁶ represents OH, NH₂, or SH;
R⁵¹ and R⁵² independently represent COOH, CH₂OH, CH₂COOH, COOR, CH₂COOR, alkyl or CO—NH₂; alternatively
R⁵¹ and R⁵² taken together represent ═O, ═S, ═CH₂ or ═NR¹⁰;
R⁵³ represents H, halogen, cyano, C₁₋₄alkyl, C₁₋₄halogenated alkyl, NO₂, O-aryl or OR¹¹.
with the proviso that at least two of X₁, X₂, X₃ and X₄ represent a carbon atom, and when any of X₁, X₂, X₃ and X₄ represent a nitrogen atom the corresponding substituent does not exist.

2. A compound of claim 1 wherein
R¹ represents OH or COOH;
R²⁰ represents H;
R⁵¹ and R⁵² taken together form ═O; and
X₁, X₂, X₃, and X₄ represent C.

3. A compound of claim 2 wherein:
R² represents halo, H, NH—CO—Ph, i-propyl, OH, OCH₃, OC₂H₅, CH(OH)COOH, O-I-propyl, SO₃H, NH₂, CH(OH)COOC₁₋₂alkyl, CH₃, NO₂ or Ph;
R³ represents H, OH, NH₂ OC₁₋₄alkyl, C₁₋₄alkyl, NHCH₃, O—(CH₂)₁₋₃—OCO—C_{1- 2}alkyl, NH—C(O)C₁₋₂alkyl, O—(CH₂)₁₋₂—CO—NH₂, Ph, NHCOCF₃, N=CH—N(CH₃)₂, O—CH₂—CO—NH—(CH₂)₁₋₃—Ph, 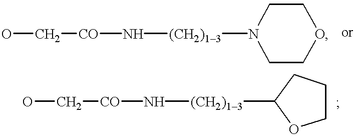
R⁴ represents H, C₁₋₄alkyl, halogen, i-propyl, OH, NH₂ 3-nitro-phen-1-yl, NH—CO—CH₃, CH₂—NH—(CH₂)₃—Ph, 2,4-difluoro-phen-1-yl, NHCOCF₃, benzo[1,3]dioxol-5-yl, 4—Carbamimidoyl-phenylazo, 3-Hydroxy-4-carboxyl-phenylsulfanyl; 1,3-Dioxo-indan-2-yl, or toluene-4-sulfonylamino;
R⁵ represents H or OH;
alternatively, R² and R³, R³ and R⁴, or R⁴ and R⁵ can be taken together to form 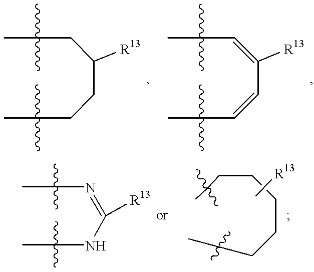
R⁶ represents H;
R⁷ represents C(═NH)—NH₂ or NH—C(═NH)—NH₂;
R⁸ represents H or halogen; and
R⁹ represents H.

4. A compound of claim 3 wherein
R² represents halo, H, NH—CO—Ph, i-propyl, OH, CH₃, or NO₂;
R³ represents H, OH, NH₂ OC₁₋₂alkyl, C₁₋₄alkyl, O—(CH₂)₁₋₃—OCO—C₁₋₂alkyl, NH—C(O)CH₃, O—CH₂—CO—NH₂, Ph, NHCOCF₃, N═CH—N(CH₃)₂, O—CH₂—CO—NH—(CH₂)₂—Ph;
R⁴ represents H, CH₃, methoxy, halogen, i-propyl, 3-nitro-phen-1-yl, NHCOCF₃, benzo[1,3]dioxol-5-yl, NHCOCH₃, 4-Carbamimidoyl-phenylazo, 3-Hydroxy-4-carboxyl-phenylsulfanyl or 1,3-Dioxo-indan-2-yl;
alternatively, R² and R³, R³ and R⁴, or R⁴ and R⁵ can be taken together to form 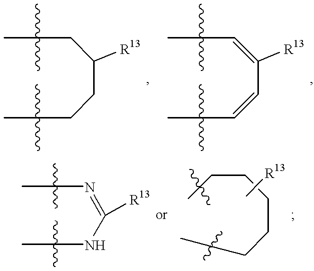
R¹³ represents C₁₋₂alkyl, OH, O(CH₂)₁₋₂—NH₂, H, or

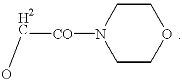

5. A compound of claim 4 wherein
R³ represents H, OH, NH₂ OC₁₋₂alkyl, C₁₋₄alkyl, O—CH₂—OCO—CH₃, NH—C(O)CH₃, O—CH₂—CO—NH₂;
R⁴represents H, CH₃, halogen, i-propyl, benzo[1,3]dioxol-5-yl, or 1,3-Dioxo-indan-2-yl;
alternatively, R² and R³, R³ and R⁴, or R⁴ and R⁵ can be taken together to form

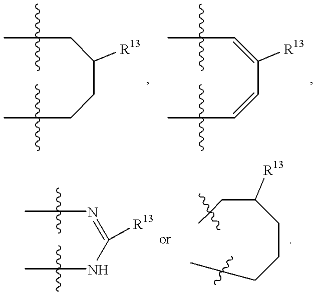

6. A compound of claim 5 wherein
R² represents H or halogen;
R³ represents H, OH or NH₂;
R⁴ represents H, CH₃, halogen or benzo[1,3]dioxol-5-yl;
R⁵ represents H; or
R³ and R⁴ or taken together to form 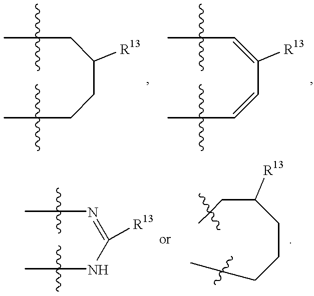
.

7. A pharmaceutical composition comprising a pharmaceutically acceptable carrier and a therapeutically effective amount of (i) a compound; or (ii) a pharmaceutically acceptable salt of a compound of claim 1.

8. A pharmaceutical composition comprising a pharmaceutically acceptable carrier and a therapeutically effective amount of a compound or a pharmaceutically acceptable salt of a compound of claim 4.

9. A method for treating or preventing a thromboembolic disorder, comprising administering to a patient in need thereof a therapeutically effective amount of a compound according to claim 4 or a pharmaceutically acceptable salt thereof.

10. A compound of claim 6, wherein the compound is selected from:
N-(4-Carbamimidoyl-phenyl)-2-hydroxy-3-iodo-5-methyl-benzamide;
3,5-Dibromo-N-(4-carbamimidoyl-phenyl)-2,4-dihydroxy-benzamide;
5-Bromo-N-(4-carbamimidoyl-phenyl)-2,4-dihydroxy-3-iodo-benzamide;
3-Hydroxy-naphthalene-2-carboxylic acid (6-guanidino-pyridin-3-yl)-amide; and
3-Hydroxy-7-methoxy-naphthalene-2-carboxylic acid (4-guanidino-phenyl)-amide.

11. A compound of claim 1 wherein
R¹ represents OH or COOH;
R²⁰ represents H;
R⁵¹ and R⁵² taken together form ═O;
X₁ represents N; and
X₂, X₃, and X₄ represent C.

12. A compound of claim 1 wherein
R² represents halo, H, NH—CO—Ph, i-propyl, OH, CH₃, NO₂ or Ph;
R³ represents H, OH, NH₂ OC₁₋₄alkyl, C₁₋₄alkyl, O—(CH₂)₁₋₃—OCO—C₁₋₂alkyl, NH—C(O)C₁₋₂alkyl, O—(CH₂)₁₋₂—CO—NH₂, Ph, NHCOCF₃, N═CH—N(CH₃)₂, O—CH₂—CO—NH—(CH₂)₁₋₃—Ph, 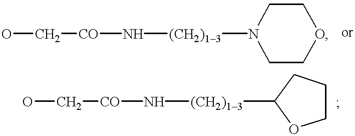
R⁴ represents H, C₁₋₄alkyl, halogen, i-propyl, OH, NH₂ 3-nitro-phen-1-yl, NH—CO—CH₃, CH₂—NH—(CH₂)₃—Ph, 2,4-difluoro-phen-1-yl, NHCOCF₃, benzo[1,3]dioxol-5-yl, 4-Carbamimidoyl-phenylazo, 3-Hydroxy-4-carboxyl-phenylsulfanyl; 1,3-Dioxo-indan-2-yl, or toluene-4-sulfonylamino;
R⁵ represents H or OH;
alternatively, R² and R³, R³ and R⁴, or R⁴ and R⁵ can be taken together to form 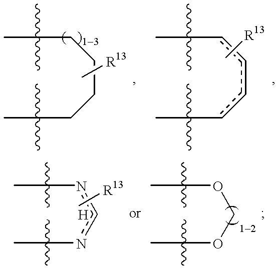
R⁶ represents H;
R⁷ represents C(═NH)—NH₂ or NH—C(═NH)—NH₂;
R⁸ represents H or halogen; and
R⁹ represents H.

13. A compound of claim 12 wherein
R² represents halo, H, NH—CO—Ph, i-propyl, OH, CH₃, or NO₂;
R³ represents H, OH, NH₂OC₁₋₂alkyl, C₁₋₄alkyl, O—(CH₂)₁₋₃—OCO—C₁₋₂alkyl, NH—C(O)CH₃, O—CH₂—CO—NH₂, Ph, NHCOCF₃, N═CH—N(CH₃)₂, O—CH₂—CO—NH—(CH₂)₂—Ph;
R⁴ represents H, CH₃, methoxy, halogen, i-propyl, 3-nitro-phen-1-yl, NHCOCF₃, benzo[1,3]dioxol-5-yl, NHCOCH₃, 4-Carbamimidoyl-phenylazo, 3-Hydroxy-4-carboxyl-phenylsulfanyl or 1,3 -Dioxo-indan-2-yl; alternatively, R² and R³, R³ and R⁴, or R⁴ and R⁵ can be taken together to form 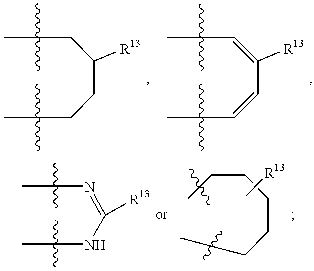
R¹³ represents C₁₋₂alkyl, OH, O(CH₂)₁₋₂—NH₂, H, or 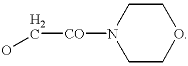
.

14. A compound of claim 13 wherein
R³ represents H, OH, NH₂ OC₁₋₂alkyl, C₁₋₄alkyl, O—CH₂—OCO—CH₃, NH—C(O)CH₃, O—CH₂—CO—NH₂;
R⁴ represents H, CH₃, halogen, i-propyl, benzo[1,3]dioxol-5-yl, or 1,3-Dioxo-indan-2-yl;
alternatively, R² and R³, R³ and R⁴, or R⁴ and R⁵ can be taken together to form 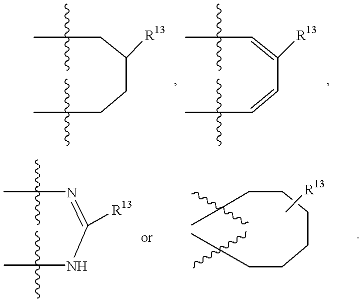
.

15. A compound of claim 14 wherein
R² represents H or halogen;
R³ represents H, OH or NH₂;
R⁴ represents H, CH₃, halogen or benzo[1,3]dioxol-5-yl;
R⁵ represents H; and
R³ and R⁴ or taken together to form 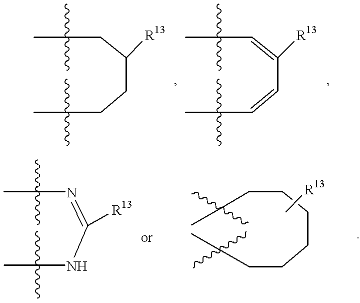
.

16. A pharmaceutical composition comprising a pharmaceutically acceptable carrier and a therapeutically effective amount of a compound or a pharmaceutically acceptable salt of a compound of claim 10.

17. A pharmaceutical composition comprising a pharmaceutically acceptable carrier and a therapeutically effective amount of a compound according to claim 13 or a pharmaceutically acceptable salt thereof.

18. A method for treating or preventing a thromboembolic disorder, comprising administering to a patient in need thereof a therapeutically effective amount of a compound according to claim 13 or a pharmaceutically acceptable salt thereof.

19. A method for treating cancer in mammals comprising administering a therapeutically effective amount of a compound according to claim 13.

20. A process for selectively acylating an amino group, said process comprising treating a molecule comprising an amino group with an acylating agent in the presence of an acetamide to yield a compound with an acylated amino group.

21. A process of claim 20 wherein the amino group is selectively acylated in the presence of another acylatable group.

22. A process of claim 21 wherein the acylatable group is selected from an optionally substituted amino ketone, alkyl amidino, alkyl guanidino, C(═NH)NH—NH₂, aryl—(CH₂)₀₋₄—NHR¹⁰, amidino and guanidino.

23. A process of claim 22 wherein the acylating agent comprises an acid halide group.

24. A process of claim 23 wherein the acetamide is an alkyl or dialkyl acetamide.

25. A process of claim 24 wherein the acetamide is selected from a group consisting of DMA, diethyl acetamide, dimethyl propionamide, diethyl propionamide and N-methylpyrrolidinone.

26. A process of claim 25 wherein the process is carried out at a temperature ranging from about 25° C. to about 50° C.

27. A process of claim 26 wherein the acylating agent is a protected salicylic acid chloride selected from acetic acid 2-chlorocarbonyl-phenyl ester and 2-benzyloxy-benzoyl chloride.

28. A method for treating or preventing a cancer related disorder, comprising administering to a patient/mammal in need thereof a therapeutically effective amount of a compound of claim 1 or a pharmaceutically acceptable salt thereof.

29. A method for treating or preventing a cancer related disorder, comprising administering to a patient/mammal in need thereof a therapeutically effective amount of a compound of claim 3 or a pharmaceutically acceptable salt thereof.

30. A method for treating or preventing a cancer related disorder, comprising administering to a patient/ mammal in need thereof a therapeutically effective amount of a compound of claim 12 or a pharmaceutically acceptable salt thereof.

31. A method for treating or preventing a cancer related disorder, comprising administering to a patient/ mammal in need thereof a therapeutically effective amount of a compound of claim 15 or a pharmaceutically acceptable salt thereof.
